(19) United States
(12) Patent Application Publication
(10) Pub. No.: US 2024/0148944 A1
(43) Pub. Date: May 9, 2024

(54) THERAPEUTIC COMPOSITIONS AND METHODS RELATED TO EXOSOME ELUTING STENTS

(71) Applicant: North Carolina State University, Raleigh, NC (US)

(72) Inventors: Ke Cheng, Raleigh, NC (US); Shiqi Hu, Raleigh, NC (US)

(21) Appl. No.: 18/549,789

(22) PCT Filed: Mar. 10, 2022
(86) PCT No.: PCT/US22/19768

§ 371 Date: Sep. 8, 2023

Related U.S. Application Data
(60) Provisional application No. 63/159,537, filed on Mar. 11, 2021.

(51) Int. Cl. A61L 31/00; A61L 31/08; A61L 31/16; A61L 33/00; A61L 33/18

(52) U.S. Cl. CPC A61L 31/005; A61L 31/08; A61L 31/16; A61L 33/0017; A61L 33/18; A61L 2300/412; A61L 2300/42; A61L 2300/626; A61L 2300/64

(57) ABSTRACT

The present disclosure provides compositions and methods relating to the use of stents treated with therapeutic biologics for the treatment of cardiovascular diseases and conditions. In particular, the present disclosure provides novel compositions and methods for conjugating therapeutic extracellular vesicles to a stent to not only regulate vascular remodeling and inflammation, but also promote the regeneration of the injured tissue.

### CROSS REFERENCE TO RELATED APPLICATIONS

This application claims priority to and the benefit of U.S. Provisional Patent Application No. 63/159,537 filed Mar. 11, 2021, which is incorporated herein by reference in its entirety for all purposes.

### FIELD

The present disclosure provides compositions and methods relating to the use of stents treated with therapeutic biologics for the treatment of cardiovascular diseases and conditions. In particular, the present disclosure provides novel compositions and methods for conjugating therapeutic extracellular vesicles to a stent to not only regulate vascular remodeling and inflammation, but also promote the regeneration of the injured tissue.

### BACKGROUND

The most common cause of peripheral artery disease is atherosclerosis. Markers of inflammation and thrombosis, elevated lipoprotein and homocysteine levels, and chronic kidney disease are also associated with peripheral artery disease. Angioplasty and stent placement are major approaches to open blocked arteries. Bare metal stents (BMS) induce proliferative and inflammatory reactions mainly because of their low biocompatible as foreign materials, which causes neointimal hyperplasia and following in-stent restenosis (ISR). Anti-proliferative drug-eluting stents (DES) have been widely applied to prevent ISR currently. However, ISR, a reparative process resembling wound healing, remains a major issue limiting the long term efficacy. Recent studies have been focusing on the development of polymer-free stent to avoid the inflammatory issues caused by the polymer, on functional coating of DES for capture of circulating endothelial progenitor cells and fast endothelization, and on novel metal materials to prevent restenosis and thrombosis.

Exosomes derived from mesenchymal stem cells (MSCs) are known to ameliorate inflammation and promote endothelial proliferation and migration, which favor the reendothelialization process. Injection of exosomes secreted from MSCs (MSC-XOs) has shown promise in treating ischemic injury such as hindlimb ischemia, myocardial infarction and renal ischemia injury. MSC-XOs have been demonstrated to be a promising experimental therapeutic for inflammatory and degenerative conditions and used in numerous clinical trials for immunomodulation and tissue regeneration, including a phase II/III clinical trial to ameliorate chronic kidney disease and a phase I/II clinical trial to treat ischemic stroke. In addition, therapeutic pro-angiogenesis effects of MSC-XOs in ischemic diseases have been studied. Therefore, there is a need to develop a stent with a coating of biologics that could not only regulate vascular remodeling and inflammation but also promote the regeneration of the injured tissue.

### SUMMARY

Embodiments of the present disclosure include a stent device comprising a plurality of extracellular vesicles conjugated to its surface with a chemical linker. In accordance with these embodiments, the plurality of extracellular vesicles includes any naturally-occurring and/or engineered exosomes, microvesicles, and/or liposomes. The extracellular vesicles conjugated to the therapeutic stents of the present disclosure can include various therapeutic agents (e.g., microRNA, biologic drugs, phospholipids, therapeutic small molecules, and the like) as cargo for the treatment of cardiovascular diseases such as ischemia, stenosis, and restenosis.

In some embodiments, the plurality of extracellular vesicles are derived from adult stem cells, induced pluripotent stem cells, and/or embryonic stem cells. In some embodiments, the plurality of extracellular vesicles are derived from mesenchymal stem cells (MSCs), cardiac stem cells (CSCs), cardiac progenitor cells (CPCs), cardiosphere-derived cells (CDCs), hematopoietic stem cells (HSCs), and/or hematopoietic progenitor cells (HPCs).

In some embodiments, the plurality of extracellular vesicles comprise therapeutic small molecules, proteins, polypeptides, peptides, nucleic acids, polynucleotide molecules, lipid-based therapeutics, and the like. In some embodiments, the plurality of extracellular vesicles comprise one or more therapeutic microRNAs (miRNAs) selected from the group consisting of hsa-let-7c-5p, hsa-let-7b-5p, hsa-let-7a-5p, hsa-miR-100-5p, hsa-miR-99a-5p, hsa-let-7f-5p, hsa-miR-23b-3p, hsa-miR-23a-3p, hsa-let-7i-5p, hsa-let-7g-5p, hsa-miR-10a-5p, hsa-miR-99b-5p, hsa-miR-148a-3p, hsa-miR-191-5p, hsa-miR-26a-5p, hsa-miR-1290, hsa-miR-320a-3p, hsa-miR-320b, hsa-miR-143-3p, hsa-miR-152-3p, hsa-miR-125b-5p, hsa-let-7d-5p, hsa-miR-320c, hsa-miR-3184-3p, hsa-miR-423-5p, and any combinations thereof. In some embodiments, the therapeutic agent comprises an anti-platelet drug and/or a regenerative factor.

In some embodiments, the liposomes comprise saturated and unsaturated fatty acid chains suitable for lipid particles. In some embodiments, the fatty acid chains are selected from the group consisting of 1,2-Distearoyl-sn-glycero-3-phosphoethanolamine (DSPE); 1,2-Distearoyl-sn-glycero-3-phosphocholine (DSPC); 1-palmitoyl-2-oleoyl-sn-glycero-3-phosphocholine (POPC); N-(2,3-dioleoyloxy) propyl)-N,N,N-triethylammonium chloride (DOTAP); and 3-(N—(N′, N′-dimethylaminoethane)-carbamoyl) cholesterol (DC-Chol).

In some embodiments, the plurality of extracellular vesicles comprise an average size of about 50 nm to about 500 nm. In some embodiments, the device comprises about 10⁵ to about 10¹⁰ extracellular vesicles per mm² of the device, which are conjugated to the surface of the device. In some embodiments, the device comprises about 10⁸ to about 10⁹ extracellular vesicles per mm² of the surface of the device.

In some embodiments, a portion of the linker is sensitive to a cleavage agent. In accordance with this embodiment, the cleavage agent is capable releasing the plurality of extracellular vesicles from the device upon exposure to the cleavage agent. In some embodiments, the linker comprises a reactive oxygen species (ROS)-sensitive portion, and the cleavage agent is an ROS.

In some embodiments, the linker comprises a phospholipid-polymer conjugate. In accordance with these embodiments, the plurality of extracellular vesicles are conjugated to the device via the phospholipid-polymer conjugate. In some embodiments, the phospholipid-polymer conjugate is inserted into the lipid membranes of the plurality of extracellular vesicles. In some embodiments, the phospholipid-polymer conjugate comprises 1,2-Distearoyl-sn-glycero-3-phosphoethanolamine-Poly(ethylene glycol) (DSPE-PEG) or any related phosphatidylcholine. In some embodiments, the PEG comprises a molecular weight of about 2,000 to about 20,000.

In some embodiments, the linker comprises thioether, alkyl selenide, telluride, alkyl diselenide, arylboronic ester, carboxyphenylboronic acid, thioketal, polysaccharide, aminoacrylate, oligoproline, and/or peroxalate ester.

In some embodiments, the surface of the device is functionalized. In some embodiments, the functionalization includes hydroxylation and/or silanization to generate chemically active groups, such as amino, hydroxyl, thiol and/or carboxy groups. In some embodiments, portion of the linker sensitive to the cleavage agent is conjugated to the device via the chemically active groups.

Embodiments of the present disclosure also include a method of treating stenosis, restenosis, and/or ischemic injury. In accordance with these embodiments, the method includes implanting any of the stents described above (comprising the conjugated extracellular vesicles) into a blood vessel of a subject.

In some embodiments, the ischemic injury comprises myocardial infarction, peripheral artery disease, stroke, mesenteric ischemia and/or renal ischemia. In some embodiments, the stent treats the stenosis or restenosis by increasing endothelial cell proliferation and/or inhibiting smooth muscle cell migration. In some embodiments, the stent treats the ischemia by increasing tissue regeneration.

### BRIEF DESCRIPTION OF THE DRAWINGS

FIGS. 1A-1F: Fabrication and characterization of EES. (a) Schematic illustration of the EES fabrication process. In brief, from bare metal stent (BMS) to exosome eluting stent (EES), the preparation process includes hydroxylation, silanization, reactive oxygen species (ROS) responsive linker, DSPE layer and exosome layer. PEG₅₀₀₀ serves as a spacer to reduce steric hindrance and improves flexibility of DSPE. DSPE is diacyl lipid and it has been widely used for the exosome membrane insertion. (b) Size distribution of MSC derived exosomes (MSC-XOs) by NanoSight. (c) Representative TEM image of MSC-XOs. Scale bar, 500 nm. d-e) TOF-SIMS spectrometry showed the surface signal of Cr from BMS (d) and C₅H₁₂N⁺ and C₃H₆NO₂⁺ signal from EES (e). (f) Representative SEM images of EES and BMS at different magnifications. The red squares are enlarged images.

FIGS. 2A-2H: In vitro ROS-trigged exosome release and biocompatibility of EES. (a) Schematic illustration of ROS responsive EES. Upon exposure to H₂O₂, the aryl boronic ester group is oxidized and subsequently hydrolyzed to unmask a phenol group. (b) Accumulative release of MSC-XOs from EES in PBS with or without H₂O₂ (100 μM or 1 mM). n=3. (c) Size distribution of MSC-XOs released from EES determined by NanoSight. (d) Representative SEM images of EES after release studies. Top image: EES in PBS; bottom image: EES in 100 μM H₂O₂. Scale bar, 20 μm. (e) MSC-XOs (DiD-labeled) release and uptake by HUVECs (at 4 h and 12 h). Scale bar, 100 μm. Representative fluorescent images and SEM images of BMS and EES after incubation with platelet-rich plasma (f) and activated monocytes (g). Adhered platelets and U937 monocytes were pointed by red arrows. (h) Quantification of adherent platelets and monocytes. n=5. P values are shown on the graph.

FIGS. 3A-3M: EES promotes the proliferation and migration of endothelial cells and inhibits the migration of smooth muscle cells. (a) Schematic illustration of the effects of EES on endothelial cells, smooth muscle cells, and injured tissue. (b) HUVEC tube formation with BMS or EES. Scale bars, 50 μm. (c) Quantification of HUVEC network nodes. (d) von Willebrand Factor (vWF) expression (green) of HUVEC with BMS or EES (exosomes were labeled with DiD (red)) were imaged via confocal microscopy, scale bars, 50 μm, and (e) Quantification of relative vWF expression. (f) CCK-8 cell proliferation assay on HUVEC co-cultured with BMS or EES for 48-h. (g) Representative images of in vitro endothelialization of HUVEC on BMS and EES. Scale bars, 20 μm. (h) Quantification of HUVEC coverage on the edge of the stent strut. (i) With BMS or EES in the lower chamber, trans-well migrated smooth muscle cells (SMCs) were stained by crystal violet, scale bars, 50 μm. (j) Quantification of migrated SMCs. (k) Representative microscopic images showing α-SMA (green) expression in SMCs co-cultured with BMS or EES. Scale bars, 100 μm. (1) Corresponding quantification. m, CCK-8 cell proliferation assay of SMC co-cultured with BMS or EES for 48-h. n=5. P values are shown on the graphs.

FIGS. 4A-4J: Stenting in the abdominal aorta of rats. (a) Schematic illustrating stent placement in the rat abdominal aorta. To demonstrate the therapeutic effects of EES towards ischemia, hindlimb ischemia or renal ischemia-reperfusion injury was induced right before placing stents. (b) SEM images of aorta with BMS or EES deployed on day 7. (c) PCR array revealing thrombosis-related gene expression in the stented vessels from sham, BMS-, or EES-stented animals. Values in EES and BMS groups were normalized to the values from the sham group. n=3. P values<0.05 are considered significant and shown in the graph. (d) Representative elastin trichrome (ET) and hematoxylin and eosin (H&E) staining of stented aortas 28 days after stent deployment. The sample was evaluated via morphometric analysis and semi-quantitative histopathologic evaluation. Lumen area (inner area), IEL (internal elastic lamina), and EEL (external elastic lamina) were outlined by yellow lines on ET images. Areas of vessel wall injury in media and adventitia outlined by green lines characterized by loss of black elastic fiber staining and increased connective tissue (blue staining) within media and adventitia. (e) Quadrant mural inflammation was analyzed and scored by the infiltration of inflammatory cells in neointima, media and strut-centered area respectively from H&E staining. The asterisks indicate struts that were fully covered. Scale bar, 1 mm. Quantification of I neointimal area, (f) average neointima thickness, (g) percent area stenosis, (h) vessel wall injury score, (i) inflammation score and (j) strut coverage in BMS, DES and EES groups. n=5. P values are shown on the graphs.

FIGS. 5A-5G: Neointimal formation with different stents. (a) Representative confocal images showing α-SMA expression around struts. n=6. (b) Quantification of α-SMA expression in the intima compared to the media. (c) GluT1 expressions in the intimal area. n=6. (d) Quantification of the density of intimal neovascularization based on GluT1 expression. (e) Quantification of the relative intimal GluT1 expression. (f) CD31 staining showing endothelial cells. (g) Quantification of the relative intimal CD31 expression. Scale bar, 100 μm. n=5. P values are shown on the graphs.

FIGS. 6A-6F: Local inflammation- and immuno-modulation effects of stent implantation. (a) Dihydroethidium (DHE) staining of aortas and (b) Quantification of ROS levels in aortas of ApoE^{-/-} rats without stenting, with BMS, DES or EES were measured using the ROS/RNS assay kit. Scale bar, 100 μm. n=5. (c) Heat map of PCR array of stented aortas. (d) Corresponding quantification of the expression of genes in the PCR array. n=4. (e) Representative CD68 (green) and CD206 (red) double staining of aortas from different groups. Scale bar, 100 μm. (f) Corresponding quantification of the numbers of CD68 and CD206 positive cells (left Y-axis), and the ratio of them (right Y-axis). n=5 animals. P values are shown on the graphs.

FIGS. 7A-7J: Restoration of blood flow and muscle repair in the ischemic limbs of ApoE^{-/-} rats after EES treatment. (a) Representative laser Doppler perfusion images taken on various time points after ischemia and stenting procedure. (left: nonischemic leg; right: ischemic leg). (b) Quantitative analysis of hindlimb blood perfusion as indicated by ischemic/nonischemic ratio. n=6. (c) Representative H&E staining of nonischemic leg and ischemic leg of each group. Scale bar, 100 μm. n=6. (d) Representative Dystrophin (green) and MHC-II (red) double staining images of legs from different groups showing the reconstruction and inflammation of each leg sample. Scale bar, 50 μm. n=6. (e-f) Quantitative analysis of MHC-II+ cells and mean cross-sectional fiber area according to the morphology of Dystrophin. (g) Immunohistochemistry of CD31 expression. Scale bar, 100 μm. h) Representative immunofluorescent images showing CD31 (green) and Ki67 positive cells (red). They overlay (yellow) of CD31 and Ki67 means proliferating endothelial cells. Scale bar, 100 μm. (i-j) Quantitative analysis of CD31⁺ and Ki67⁺ cells. n=6. P values are shown on the graphs.

FIG. 8: Chemical structure of DSPE-conjugated stents.

FIGS. 9A-9C: Characterization of MSC-XOs. (a) Western blot of MSC-XOs verified common exosomal markers, including Alix, TSG101 and CD81. (b) Top twenty-five miRNAs expressed in MSC-XOs. (c) Top ten proteins identified in MSC-XOs.

FIG. 10: XPS spectrum of bare metal, NH₂—, DSPE- and exosomes-conjugated surfaces. Stainless steel disks were used instead of stents as the surface area of coronary stents is too small for XPS test. From top to bottom, bare metal, —OH (binding energy change of Fe 2p 3/2 from 710.93 to 706.47), -ATPES coated (enhanced N is signal), -PEG-DSPE coated (enhanced C is signal and decreased N is signal), and -MSC-XOs coated surface (enhanced N signal and absence of Cr 2p 3/2 signal).

FIG. 11: In vitro endothelial coverage on BMS and EES after 4 hours of incubation. Scale bar, 200 μm.

FIGS. 12A-12E: Effects of EES and BMS on HCAEC. (a) VE-cadherin and vWF double staining of HCAEC co-cultured with BMS, DES or EES. Scale bars, 100 μm. (b) Quantification of vWF expression in each group. (c) CCK-8 cell proliferation assay on HCAEC co-cultured with BMS, DES or EES for 48 h. (d) HCAEC tube formation in vitro with BMS, DES or EES. Scale bars, 200 μm. (e) Quantification of HCAEC network nodes. n=5. P values are shown on the graphs

FIG. 13: Schematic illustration of the trans-well migration experiments used in the present disclosure.

FIGS. 14A-14C: Biodistribution of DiR-labeled MCS-XOs released from EES in rats with ischemic renal injury. (a) Schematic illustration of the layout of organs and stented aortas for IVIS imaging. (b) Organs and aortas from sham rats with BMS stenting, sham rats with EES stenting, and rats with renal ischemia-reperfusion injury and EES stenting 3 days after stenting. (c) Quantification of the fluorescent intensities from DiR-labeled MSC-XOs in each organ. n=3, P values are shown on the graph.

FIGS. 15A-15B: Histopathologic staining of BMS, DES and EES groups 28 days after stent deployment. (a) Elastin trichrome (ET) and (b) hematoxylin and eosin (H&E) staining. The sample was evaluated via morphometric analysis and semi-quantitative histopathologic evaluation. Lumen area (inner area), IEL (Middle) and EEL (outer boundary) were outlined by yellow lines on ET images. Areas of vessel wall injury in media and adventitia outlined by green lines characterized by loss of black elastic fiber staining and increased connective tissue (blue staining) within media and adventitia. Quadrant mural inflammation was analyzed and scored by the infiltration of inflammatory cells in neointima, media and strut-centered area respectively from H&E staining. The asterisk indicates the strut was fully covered.

FIGS. 16A-16G: EES treatment effects on renal functions and structures. (a) Hematoxylin & Eosin (H&E, scale bar, 60 μm), Masson's Trichrome (scale bar, 220 μm), Ki67 (scale bar, 100 μm) and TUNEL staining (scale bar, 100 μm) of sham, control, BMS and EES respectively on day 7. (b-e) Quantification of (b) renal tubular necrosis, (c) fibrosis degree, (d) Ki67 positive (Ki67^{pos}) cells, and (e) TUNEL^{pos} cells. (f-g) Blood samples on day 1, 7 and 28 of different groups were harvested, blood parameters, SCr and BUN were tested. BMS and EES groups were compared. n=5. P values are shown on the graphs.

FIG. 17: Oil Red O staining. Oil Red O-stained organs and cross sections of the aortic vessel reveal the neutral lipids of ApoE^{-/-} rats. Scale bar, 100 μm.

FIG. 18: H&E and Masson's Trichrome staining of vessels. Images show aortic lesions in ApoE^{-/-} rats but not in wild type Sprague Dawley (SD) rats. Scale bar, 220 μm.

FIGS. 19A-19B: CD68 expression in the limbs of rats. (a) Representative CD68 (red) double staining images of legs from different groups. Scale bar, 100 μm. (b) Quantification of CD68 expression of the ischemic limb of different groups compared to non-ischemic limbs. n=5. P values are shown on the graph.

FIG. 20: H&E staining of organs from control, BMS-, DES- and EES-stented ApoE^{-/-} rats. Inflammatory cell infiltration in the spleen, mast cells in the lung, and glomeruli in the kidney were pointed with red arrows. Scale bar, 100 μm.

### DETAILED DESCRIPTION

Drug-eluting stents implanted after ischaemic injury reduced the proliferation of endothelial cells and vascular smooth muscle cells and thus neointimal hyperplasia. However, the eluted drug also slows down the re-endothelialization process, delays arterial healing, and can increase the risk of late restenosis. In the present disclosure, experiments were conducted to test the hypothesis that stents could be ideal carriers to deliver therapeutic extracellular vesicles (e.g., exosomes) to ischemic tissue. Synergistically, the exosome coating could improve the biocompatibility of the stent to promote local vascular healing after stent deployment. To those ends, a bioresponsive exosome-eluting stent (EES) was designed by taking advantage of the elevated level of reactive oxygen species (ROS) from the mechanical injury during stent deployment. ROS is also reportedly a biomarker in vascular diseases including atherosclerosis. Studies have demonstrated that the increase in oxidative stress and inflammatory response lead to neointimal formation, vascular smooth muscle cell (SMC) proliferation, and adverse extracellular matrix deposition. Those pathological events subsequently lead to restenosis.

Embodiments of the present disclosure demonstrate that stents releasing exosomes derived from mesenchymal stem cells in the presence of reactive oxygen species enhanced vascular healing in rats with renal ischaemia-reperfusion injury, promoting endothelial-cell tube formation and proliferation, and impairing the migration of smooth muscle cells. Compared with drug-eluting stends and bare-metal stents, the exosome-coated stents accelerated re-endothelialization and decreased in-stent restenosis 28 days after implantation. Results of experiments also demonstrated that exosome-eluting stents implanted in the abdominal aorta of rats with unilateral hindlimb ischaemia regulated macrophage polarization, reduced local vascular and systemic inflammation, and promoted muscle-tissue repair. Thus, exosome-eluting stents implanted after ischaemic injury accelerated vascular healing by promoting endothelial-cell tube formation and proliferation and impairing the migration of smooth muscle cells.

Section headings as used in this section and the entire disclosure herein are merely for organizational purposes and are not intended to be limiting.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Correlated to” as used herein refers to compared to.

As used herein, the term “animal” refers to any animal (e.g., a mammal), including, but not limited to, humans, non-human primates, pigs, rodents (e.g., mice, rats, etc.), flies, and the like.

The terms “medium” and “cell culture medium” (plural, “media”) generally refer to a nutrient source used for growing or maintaining cells. As is understood by a person of ordinary skill in the art based on the present disclosure, a growth medium or cell culture medium is a liquid or gel designed to support the growth of microorganisms, cells, or small plants. Cell culture media generally comprise an appropriate source of energy and compounds which regulate the cell cycle. A typical culture medium can be composed of, but not limited to, a complement of amino acids, vitamins, inorganic salts, glucose, and serum as a source of growth factors, hormones, and attachment factors. In addition to nutrients, the medium also helps maintain pH and osmolality.

The terms “administration of” and “administering” a composition as used herein refers to providing a composition of the present disclosure to a subject in need of treatment. The compositions of the present disclosure may be administered by topical (e.g., in contact with skin or surface of body cavity), oral, parenteral (e.g., intramuscular, intraperitoneal, intravenous, ICV, intracistemal injection or infusion, subcutaneous injection, or implant), by spray, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration.

The term “composition” as used herein refers to a product comprising the specified ingredients in the specified amounts, as well as any product which results, directly or indirectly, from combination of the specified ingredients in the specified amounts. Such a term in relation to a pharmaceutical composition is intended to encompass a product comprising the active ingredient(s), and the inert ingredient(s) that make up the carrier, as well as any product which results, directly or indirectly, from combination, complexation, or aggregation of any two or more of the ingredients, or from dissociation of one or more of the ingredients, or from other types of reactions or interactions of one or more of the ingredients. Accordingly, the pharmaceutical compositions of the present disclosure encompass any composition made by admixing, e.g., exosomes and/or miRNAs of the present disclosure and a pharmaceutically acceptable carrier and/or excipient. When exosomes and/or miRNAs of the present disclosure are used contemporaneously with one or more other drugs, a pharmaceutical composition containing such other drugs in addition to the exosomes and/or miRNAs of the present disclosure are contemplated. Accordingly, the pharmaceutical compositions of the present disclosure include those that also contain one or more other active ingredients, in addition to a exosomes and/or miRNAs of the present disclosure. The weight ratio of the exosomes and/or miRNAs of the present disclosure may be varied and will depend upon the effective dose of each ingredient. Generally, an effective dose of each will be used. Combinations of exosomes and/or miRNAs of the present disclosure and other active ingredients will generally also be within the aforementioned range, but in each case, an effective dose of each active ingredient should be used. In such combinations the exosomes and/or miRNAs of the present disclosure and other active agents may be administered separately or in conjunction. In addition, the administration of one element may be prior to, concurrent to, or subsequent to the administration of other agent(s).

The term “pharmaceutical composition” as used herein refers to a composition that can be administered to a subject to treat or prevent a disease or pathological condition, and/or to improve/enhance one or more aspects of a subject's physical health. The compositions can be formulated according to known methods for preparing pharmaceutically useful compositions (e.g., exosome preparation). Furthermore, as used herein, the phrase “pharmaceutically acceptable carrier” means any of the standard pharmaceutically acceptable carriers. The pharmaceutically acceptable carrier can include diluents, adjuvants, and vehicles, as well as implant carriers, and inert, non-toxic solid or liquid fillers, diluents, or encapsulating material that does not react with the active ingredients of the invention. Examples include, but are not limited to, phosphate buffered saline, physiological saline, water, and emulsions, such as oil/water emulsions. The carrier can be a solvent or dispersing medium containing, for example, ethanol, polyol (for example, glycerol, propylene glycol, liquid polyethylene glycol, and the like), suitable mixtures thereof, and vegetable oils. Formulations containing pharmaceutically acceptable carriers are described in a number of sources which are well known and readily available to those skilled in the art. For example, Remington's Pharmaceutical Sciences (Martin E W, Remington's Pharmaceutical Sciences, Easton Pa., Mack Publishing Company, 19.sup.th ed., 1995) describes formulations that can be used in connection with the subject invention.

The term “pharmaceutically acceptable carrier, excipient, or vehicle” as used herein refers to a medium which does not interfere with the effectiveness or activity of an active ingredient and which is not toxic to the hosts to which it is administered and which is approved by a regulatory agency of the Federal or a state government or listed in the U.S. Pharmacopeia or other generally recognized pharmacopeia for use in animals, and particularly in humans. A carrier, excipient, or vehicle includes diluents, binders, adhesives, lubricants, disintegrates, bulking agents, wetting or emulsifying agents, pH buffering agents, and miscellaneous materials such as absorbents that may be needed in order to prepare a particular composition. Examples of carriers etc. include but are not limited to saline, buffered saline, dextrose, water, glycerol, ethanol, and combinations thereof. The use of such media and agents for an active substance is well known in the art.

As used herein, the term “effective amount” generally means that amount of a drug or pharmaceutical agent that will elicit the biological or medical response of a tissue, system, animal or human that is being sought, for instance, by a researcher or clinician. Furthermore, the term “therapeutically effective amount” generally means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

The term “combination” and derivatives thereof, as used herein, generally means either, simultaneous administration or any manner of separate sequential administration of a therapeutically effective amount of Compound A, or a pharmaceutically acceptable salt thereof, and Compound B or a pharmaceutically acceptable salt thereof, in the same composition or different compositions. If the administration is not simultaneous, the compounds are administered in a close time proximity to each other. Furthermore, it does not matter if the compounds are administered in the same dosage form (e.g., one compound may be administered topically and the other compound may be administered orally).

As used herein, the term “subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (e.g., a monkey, such as a cynomolgus or rhesus monkey, chimpanzee, etc.) and a human). In some embodiments, the subject may be a human or a non-human. In one embodiment, the subject is a human. The subject or patient may be undergoing various forms of treatment.

As used herein, the term “treat,” “treating” or “treatment” are each used interchangeably herein to describe reversing, alleviating, or inhibiting the progress of a disease and/or injury, or one or more symptoms of such disease, to which such term applies. Depending on the condition of the subject, the term also refers to preventing a disease, and includes preventing the onset of a disease, or preventing the symptoms associated with a disease. A treatment may be either performed in an acute or chronic way. The term also refers to reducing the severity of a disease or symptoms associated with such disease prior to affliction with the disease. Such prevention or reduction of the severity of a disease prior to affliction refers to administration of a treatment to a subject that is not at the time of administration afflicted with the disease. “Preventing” also refers to preventing the recurrence of a disease or of one or more symptoms associated with such disease.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, cell biology, genetics and protein and nucleic acid chemistry described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

### 2. COMPOSITIONS AND METHODS

Despite its wide use in vascular medicine, BMS can cause mechanical injury to the blood vessel, followed by local inflammatory response that further stimulates the migration of vascular SMCs and impedes endothelialization. DES release anti-proliferative drugs and reduce rates of restenosis and repeat revascularization compared with BMS. However, some early generations of DES were associated with higher rates of stent thrombosis than BMS particularly beyond the first few months after implantation. Furthermore, neither BMS nor DES carries biologically-active substances to directly promote tissue regeneration.

Naturally derived exosomes were largely used as therapeutic vehicles due to their safety, biocompatibility and stability. Embodiments of the present disclosure used MSC-XOs as the regenerative cargo, due to the reported role in tissue repair and excellent safety profile. The biodegradable linker used to link the therapeutic exosomes to the stent, benzeneboronic acid pinacol ester group, is highly sensitive to ROS. EES with arylboronic ester derivatives were designed to be sensitive to elevated reactive oxygen species (ROS, 50 μM˜100 μM), which is induced by both local inflammation after stenting and atherosclerosis. The numbers of exosomes coated onto stents were estimated. Assuming the diameter of exosomes is 100 nm, the cross-sectional area of exosomes would be:

Aₑₓₒ=πr²=π·(50×10⁻⁶ mm)²=7.85×10⁻⁹ mm²

The surface area of the stents varies depending on the length of the stents. Taking Boston Scientific Rebel PtCr OTW coronary stent 2.25 mmx 12 mm for example, the folded average stent profile is 1.07 mm in diameter, the surface (internal and external) would be:

Aₛₜₑₙₜ=2×πd×l=2π·1.07 mm·12 mm≈80 mm²

The number of monolayer exosomes can be coated:

n=Aₛₜₑₙₜ/Aₑₓₒ=80/7.85×10⁻⁹≈10¹⁰

The struts of stents are close to a cylinder, so the whole contact area of the deployed stent with exosomes would be larger. There also can be multiple layers of exosomes linked to the stent. Therefore, the estimated loading number of exosomes is around 10¹⁰˜10¹¹ per stent. MSC-XOs were quantified per stent by counting the number of MSC-XOs before and after EES fabrication, and it was found that 1.0˜1.5×10¹⁰ MSC-XOs were coated onto the stent. In one of the previous studies, the dose of intravenous MSC-XOs was 1×10¹²/kg mice. However, most exosomes were rapidly taken up by macrophages in the reticuloendothelial system, which greatly limits the application of systemic administration of exosomes. According to Gallet et al., the dose of exosomes by intracoronary or open-chest intramyocardial delivery was 4.1×10¹⁰ and 2.1×10¹⁰/kg respectively in Yucatan pigs. The EES system of the present disclosure could deliver 10¹⁰˜10¹¹ exosomes per stent through a minimal invasive approach, which is enough to demonstrate therapeutic effect as a local delivery device.

After that, the release profile of MSC-XOs from EES was investigated. Within 48 hours, around 40%˜60% of exosomes could be released upon ROS stimulation in vitro as compared to a 20% release under normal physical environment. This demonstrates that EES can quickly respond to elevated ROS level to release exosomes. In vivo, the release of exosomes into the circulation could be shorter as the struts of the stent are gradually covered by blood cells, vascular cells and extracellular matrix deposited by the cells.

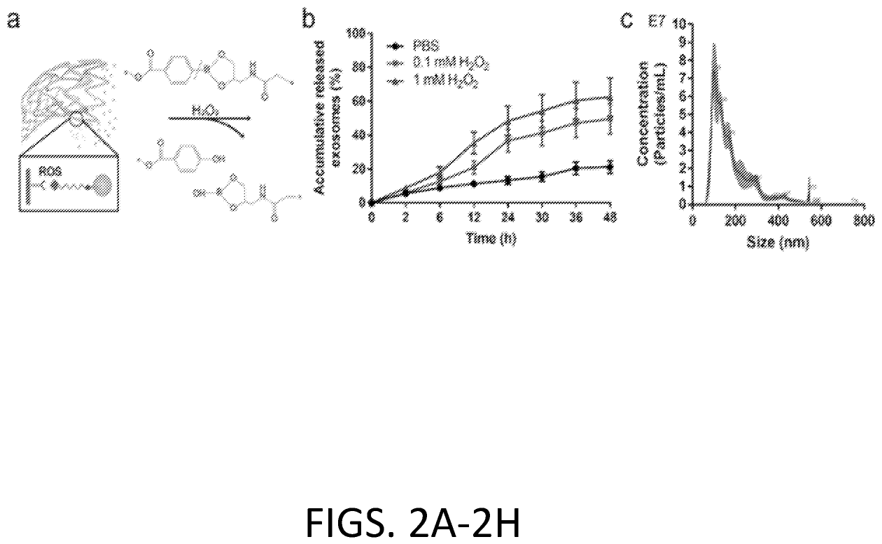

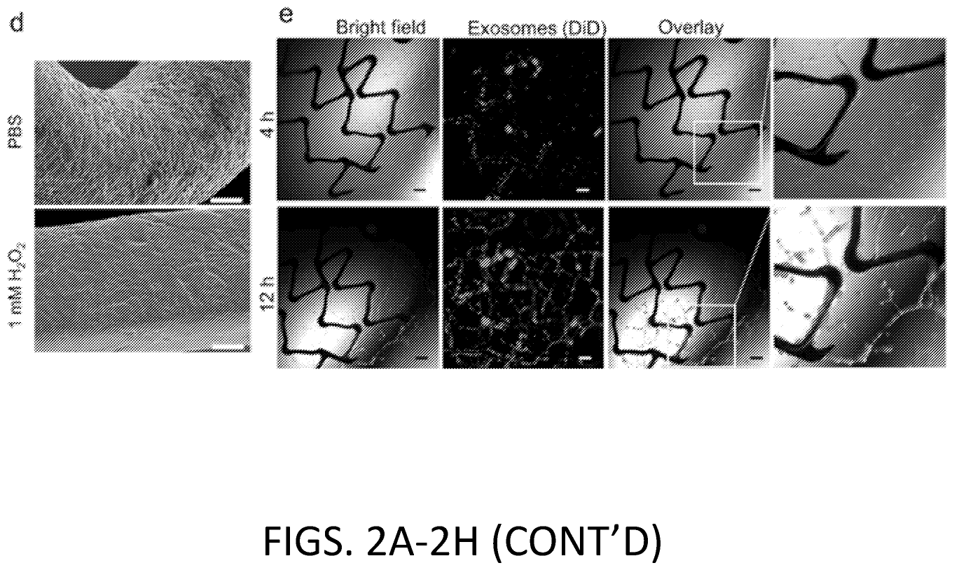

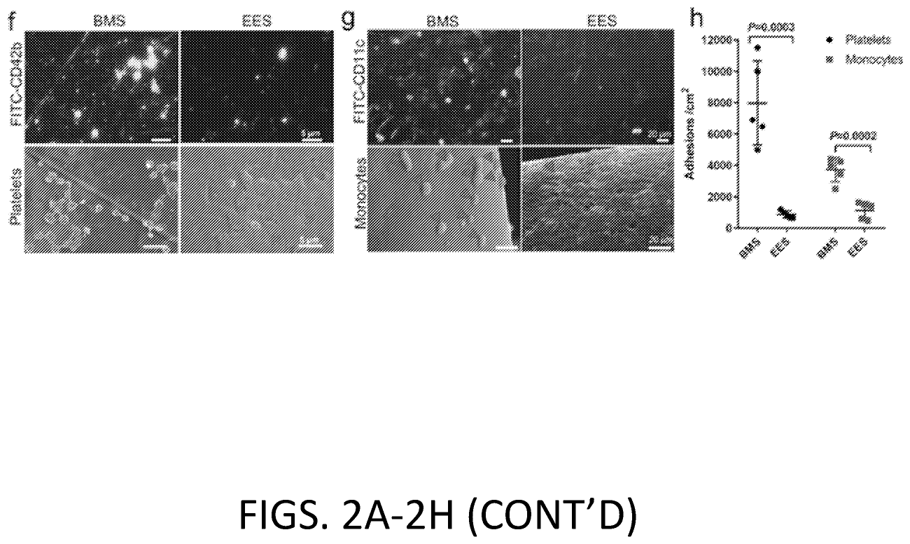

Coating with a biocompatible material is reportedly to improve the safety of medical devices. Human origin MSC-XOs were widely used in small animal studies due to their hypo-immunogenicity. Unlike stem cells, exosomes could be sterile filtered, and they would not cause abnormal tissue growth due to their nonviable property. In addition, the phospholipid bilayer of exosomes provided a superior alternative to synthetic polymer coating due to its similar composition as compared to cell membranes. The reduced adhesion of platelets and monocytes on EES as compared to BMS were confirmed (FIGS. 2F-2G).

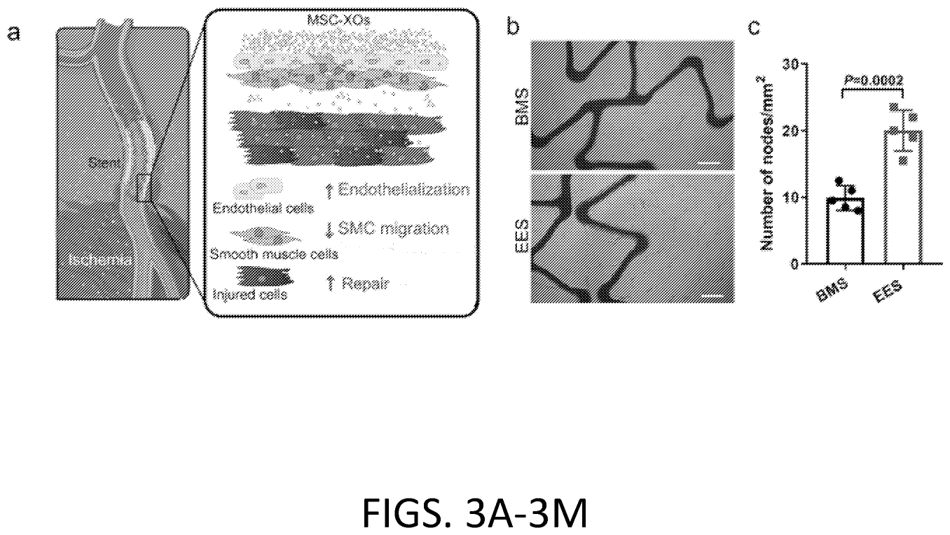

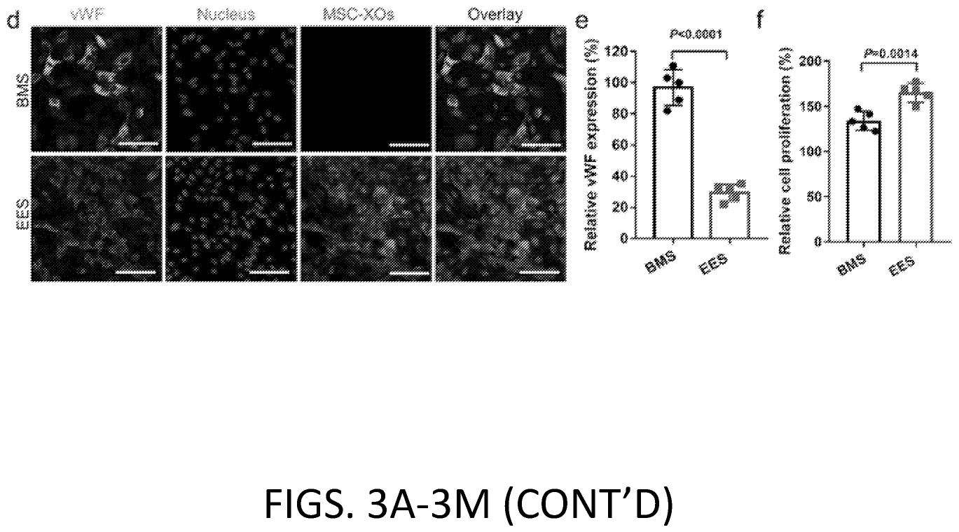

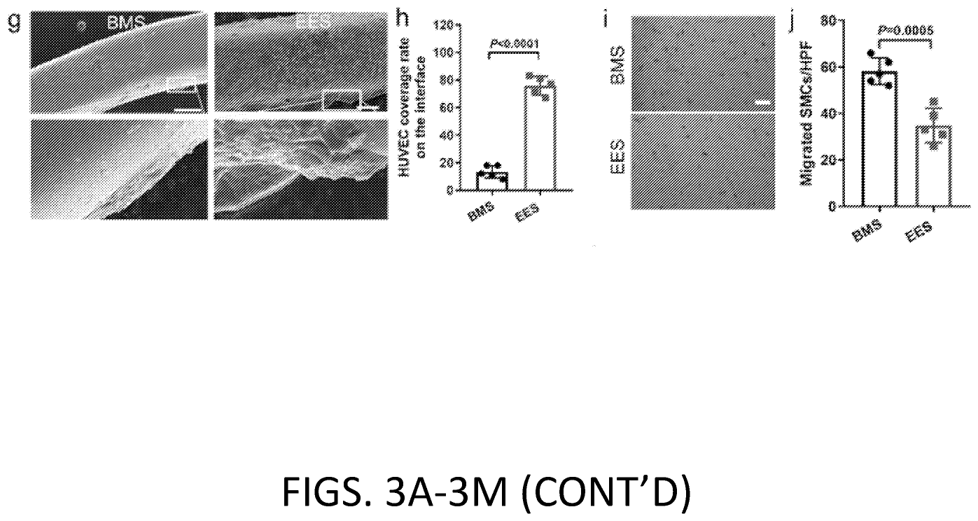

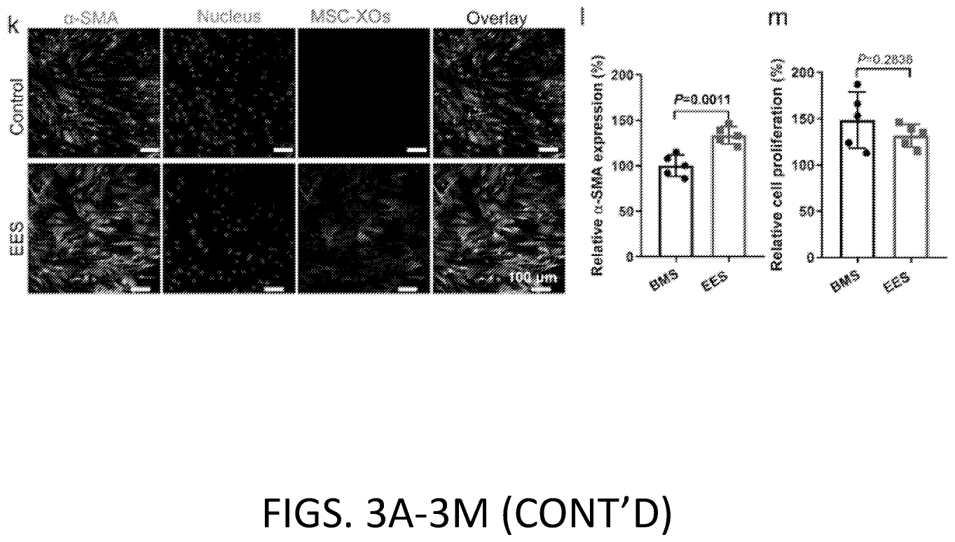

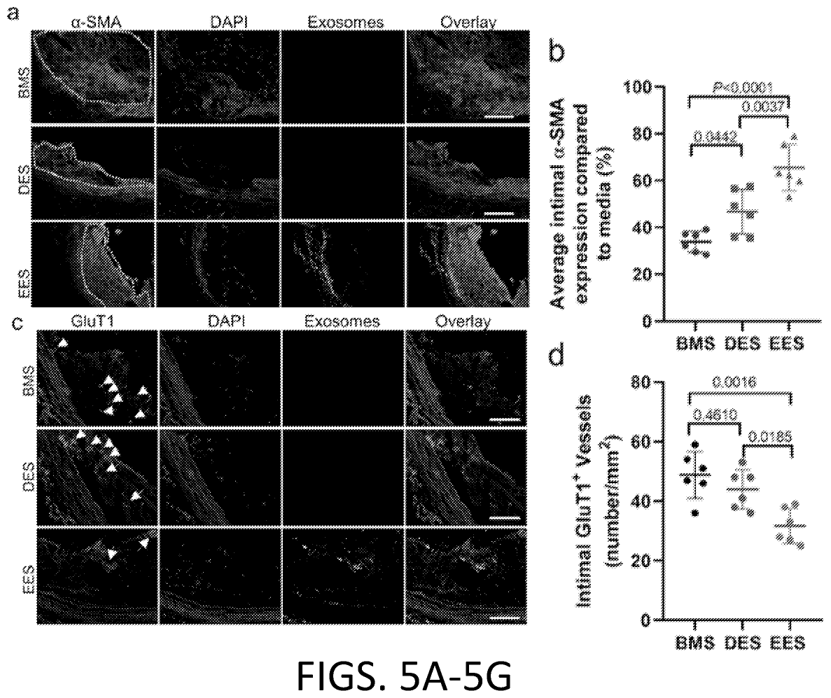

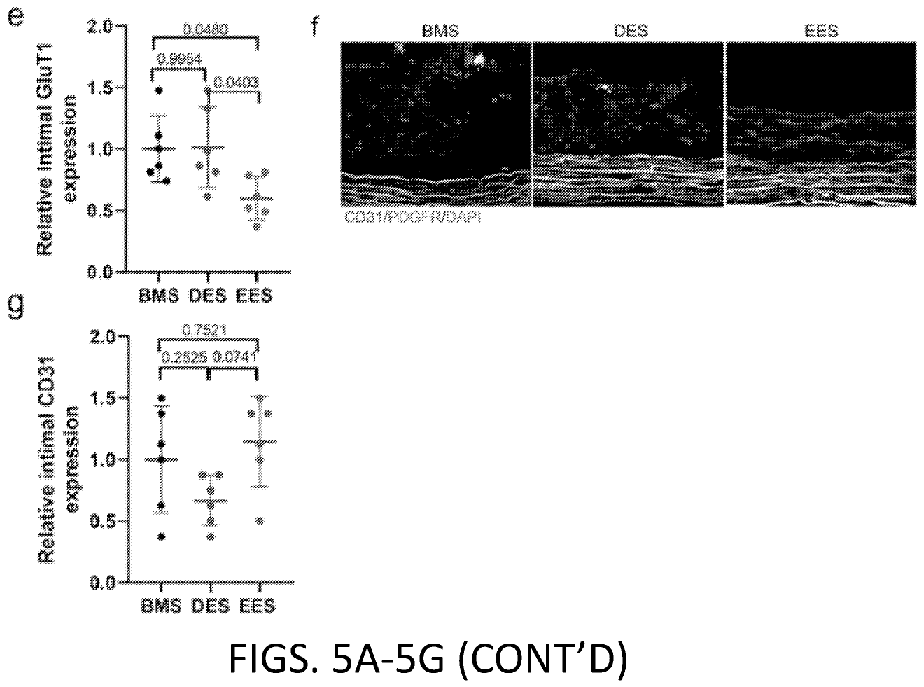

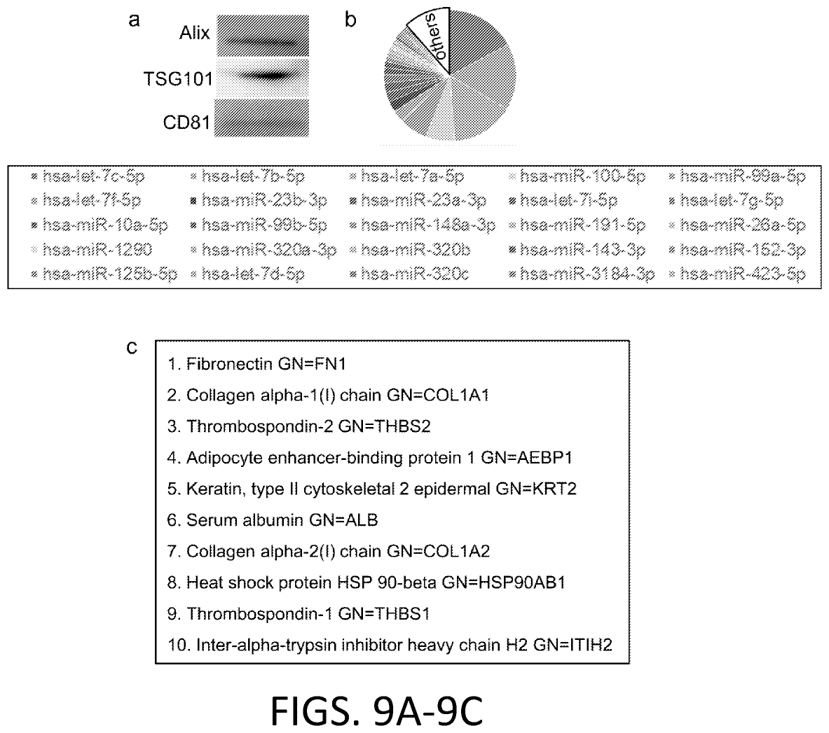

EES promoted the proliferation of ECs and their migration to the stents. Reportedly those are also observed as a benefit of MSC-XO treatment. The mechanisms underlying such effects remain elusive, but they are likely come from the microRNA cargos in MSC-XOs (FIG. 9). For example, miR-23a-3p and let-7b-5p in MSC-XOs target genes related to angiogenesis and regulate vascular repair. Besides miRNAs, abundant extracellular-associated proteins, like fibronectin and collagen α1 were identified in MSC-XOs (FIG. 9). Those exosomal proteins also play critical roles in angiogenesis. In addition, the effects of EES on the proliferation and migration of SMCs were investigated. EES favors the modulation of SMCs in a contractile phenotype with a higher expression of α-SMA as compared to BMS (FIG. 3K). This was confirmed in vivo in the ApoE^{-/-} rat model of atherosclerosis (FIG. 5A). SMCs are the primary cell type in the pre-atherosclerotic intima. The phenotypic modulation of intimal SMCs occurs in response to environmental change. The proliferation and migration of SMCs in the intima require the transition of SMCs from a contractile to a synthetic phenotype. EES didn't slow down the proliferation of SMCs as this is an important step for early-stage stent coverage (FIG. 3M).

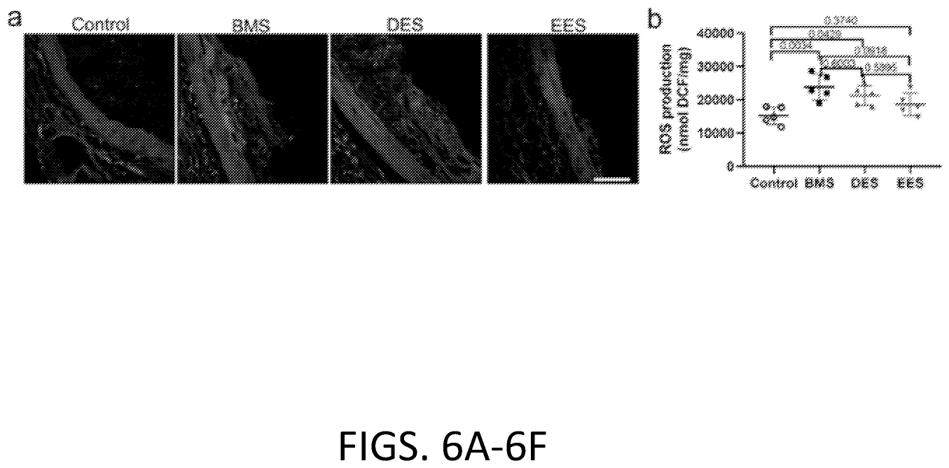

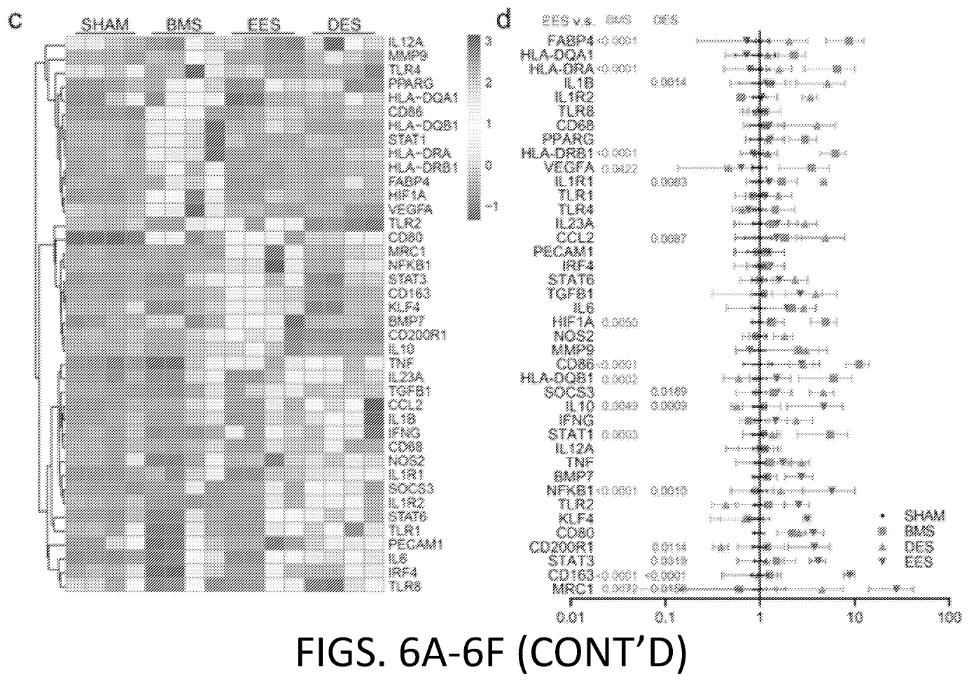

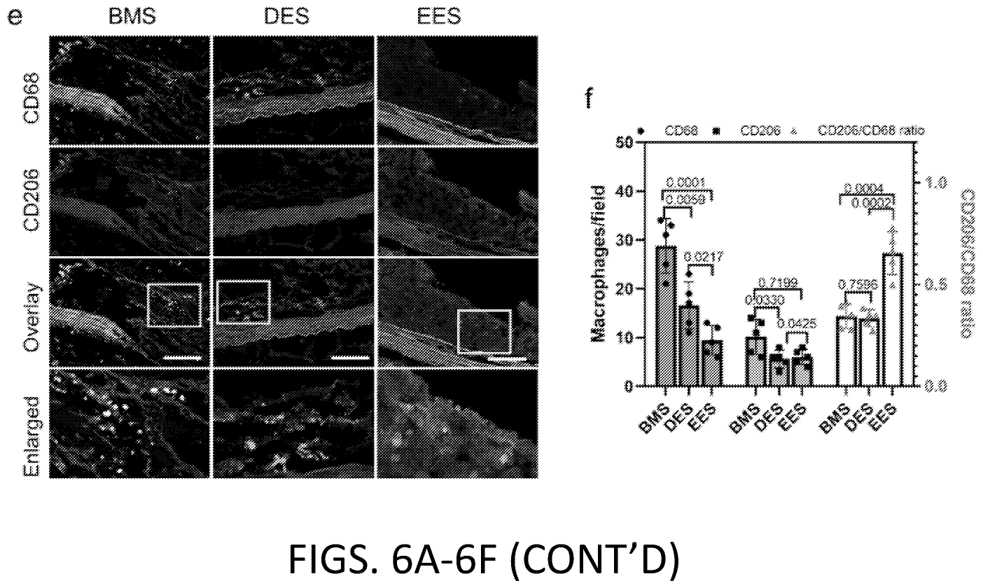

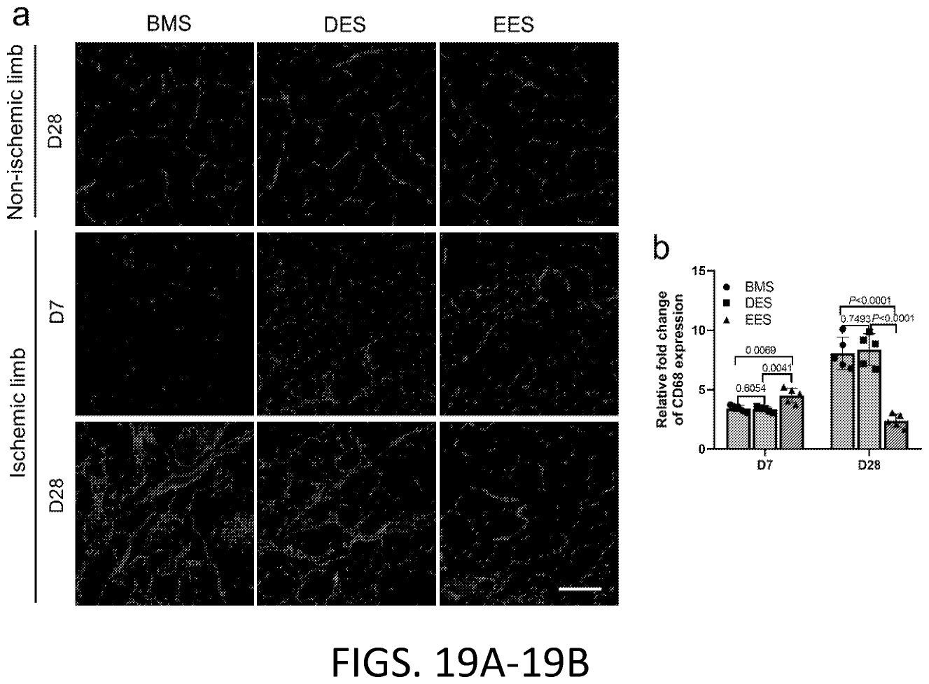

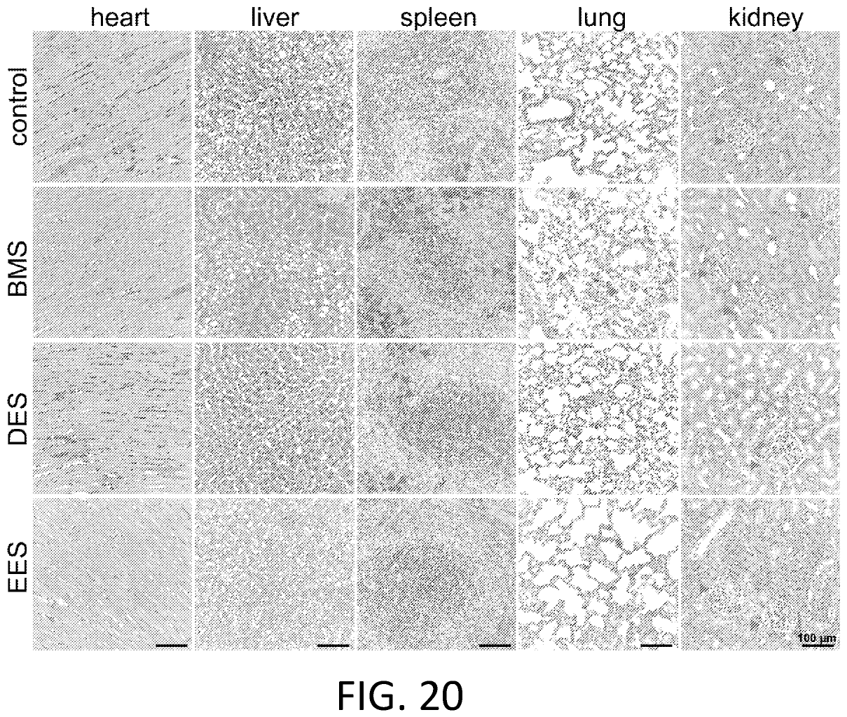

Anti-inflammation and pro-angiogenesis mechanisms underlies MSC-XO-mediated tissue repair. The inflammation and the vascular remodeling after stenting are highly related to local monocyte's behaviors. MSC-XOs were reportedly to have the ability to modulate macrophage polarization and inhibit inflammation in the tissue remodeling process. It has been well established that M1 macrophages mediated the secretion of pro-inflammatory factors and vascular smooth muscle cell migration, while M2 macrophages encourage wound healing and reendothelization. Mounting lines of evidence correlates enhanced level of anti-inflammatory M2 macrophages leads with less in-stent restenosis. In addition, M2 macrophage-derived exosomes were studied for their effects on SMC dedifferentiation and vascular repair process. Immunostaining and PCR array (FIG. 6) showed reduced inflammation and M2 macrophage polarization with EES treatment, suggesting a positive role of EES in vascular healing and remodeling. Further, the effects of MSC-XOs on major organs were investigated. As shown in FIG. 19, lipid deposition in the liver was evident in all groups. Histology of the heart and liver was found to be normal. ApoE^{-/-} rats displayed mast cell infiltration in the lung, mesangial proliferative glomerulonephritis, and splenomegaly. A decrease of inflammation in the spleen and the glomeruli of the EES group was found. The spleen is an important organ for atherosclerosis-associated immunity, and the change of inflammatory cells in the spleen with MSC-XOs (released from EES) deserves further investigation.

As summarized in Table 2, mounting lines of evidence have confirmed the advantages of DES over BMS. However, late in-stent restenosis rate of current DES still remains high (˜3%-20%) after stent implantation. Long-term follow up studies of the performance of DES are likely needed to completely understand the safety profile. In the present disclosure, the use of synthetic polymers and anti-proliferative drugs were avoided, but MSC-XOs were used as a biocompatible and bioactive coating. Given MSC-XOs have showed promise in attenuating atherosclerosis and promoting reendothelialization, they are ideal coating materials for vascular stents. In addition, MSC-XOs have been tested in a wide range of ischemia diseases, such as myocardial infarction and peripheral and renal ischemic injury.

In the present disclosure, the EES group showed a much smaller neointimal area compared to the BMS group, and a much higher strut coverage rate compared to the DES group 28 days after deployment. The histological analysis proved the functional advantage of EES. Additionally, the therapeutic effect of EES towards terminal ischemic tissue has been demonstrated in rats with renal ischemic or hindlimb ischemic injury. The present disclosure offers translational values. Given the excellent stability of exosomes, EES can be an off-the-shelf platform product to be applied in acute settings. This “biological stent” holds the potential to not only mechanically keep the vessel open but also to repair the injured tissue, which is something not accomplished by current stent products.

Thus, embodiments of the present disclosure include a biocompatible and bioactive stent that can release therapeutic exosomes under ROS. EES is free of polymer coating and anti-proliferative drugs (used in DES) and decreases the risks of thrombosis and inflammation. EES accelerates the vascular healing process via promoting endothelial proliferation and early-stage cell coverage, while reducing inflammation and SMC migration. In addition, EES treatment promotes tissue repair in renal ischemia and hind limb ischemia models via pro-angiogenesis mechanisms.

In accordance with these embodiments, the present disclosure includes a stent device comprising a plurality of extracellular vesicles conjugated to its surface with a chemical linker. In accordance with these embodiments, the plurality of extracellular vesicles includes any naturally-occurring and/or engineered exosomes, microvesicles, and/or liposomes. The extracellular vesicles conjugated to the therapeutic stents of the present disclosure can include various therapeutic agents (e.g., microRNA, biologic drugs, phospholipids, therapeutic small molecules, and the like) as cargo for the treatment of cardiovascular diseases such as ischemia, stenosis, and restenosis.

In some embodiments, the plurality of extracellular vesicles are derived from adult stem cells, induced pluripotent stem cells, and/or embryonic stem cells. In some embodiments, the plurality of extracellular vesicles are derived from mesenchymal stem cells (MSCs), cardiac stem cells (CSCs), cardiac progenitor cells (CPCs), cardiosphere-derived cells (CDCs), hematopoietic stem cells (HSCs), and/or hematopoietic progenitor cells (HPCs).

In some embodiments, the plurality of extracellular vesicles comprise therapeutic small molecules, proteins, polypeptides, peptides, nucleic acids, polynucleotide molecules, lipid-based therapeutics, and the like. In some embodiments, the plurality of extracellular vesicles comprise one or more therapeutic microRNAs (miRNAs) selected from the group consisting of hsa-let-7c-5p, hsa-let-7b-5p, hsa-let-7a-5p, hsa-miR-100-5p, hsa-miR-99a-5p, hsa-let-7f-5p, hsa-miR-23b-3p, hsa-miR-23a-3p, hsa-let-7i-5p, hsa-let-7g-5p, hsa-miR-10a-5p, hsa-miR-99b-5p, hsa-miR-148a-3p, hsa-miR-191-5p, hsa-miR-26a-5p, hsa-miR-1290, hsa-miR-320a-3p, hsa-miR-320b, hsa-miR-143-3p, hsa-miR-152-3p, hsa-miR-125b-5p, hsa-let-7d-5p, hsa-miR-320c, hsa-miR-3184-3p, hsa-miR-423-5p, and any combinations thereof. In some embodiments, the therapeutic agent comprises an anti-platelet drug and/or a regenerative factor.

In some embodiments, the liposomes comprise saturated and unsaturated fatty acid chains suitable for lipid particles. In some embodiments, the fatty acid chains are selected from the group consisting of 1,2-Distearoyl-sn-glycero-3-phosphoethanolamine (DSPE); 1,2-Distearoyl-sn-glycero-3-phosphocholine (DSPC); 1-palmitoyl-2-oleoyl-sn-glycero-3-phosphocholine (POPC); N-(2,3-dioleoyloxy) propyl)-N,N,N-triethylammonium chloride (DOTAP); and 3-(N—(N′, N′-dimethylaminoethane)-carbamoyl) cholesterol (DC-Chol).

In some embodiments, the plurality of extracellular vesicles comprise an average size of about 50 nm to about 500 nm. In some embodiments, the device comprises about 10⁵ to about 10¹⁰ extracellular vesicles per mm² of the device, which are conjugated to the surface of the device. In some embodiments, the device comprises about 10⁸ to about 10⁹ extracellular vesicles per mm² of the surface of the device.

In some embodiments, a portion of the linker is sensitive to a cleavage agent. In accordance with this embodiment, the cleavage agent is capable releasing the plurality of extracellular vesicles from the device upon exposure to the cleavage agent. In some embodiments, the linker comprises a reactive oxygen species (ROS)-sensitive portion, and the cleavage agent is an ROS.

In some embodiments, the linker comprises a phospholipid-polymer conjugate. In accordance with these embodiments, the plurality of extracellular vesicles are conjugated to the device via the phospholipid-polymer conjugate. In some embodiments, the phospholipid-polymer conjugate is inserted into the lipid membranes of the plurality of extracellular vesicles. In some embodiments, the phospholipid-polymer conjugate comprises 1,2-Distearoyl-sn-glycero-3-phosphoethanolamine-Poly(ethylene glycol) (DSPE-PEG) or any related phosphatidylcholine. In some embodiments, the PEG comprises a molecular weight of about 2,000 to about 20,000.

In some embodiments, the linker comprises thioether, alkyl selenide, telluride, alkyl diselenide, arylboronic ester, carboxyphenylboronic acid, thioketal, polysaccharide, aminoacrylate, oligoproline, and/or peroxalate ester.

In some embodiments, the surface of the device is functionalized. In some embodiments, the functionalization includes hydroxylation and/or silanization to generate functional amino groups. In some embodiments, portion of the linker sensitive to the cleavage agent is conjugated to the device via the amino functional groups.

Embodiments of the present disclosure also include a method of treating stenosis, restenosis, and/or ischemic injury. In accordance with these embodiments, the method includes implanting any of the stents described above (comprising the conjugated extracellular vesicles) into a blood vessel of a subject.

In some embodiments, the ischemic injury comprises myocardial infarction, peripheral artery disease, stroke, mesenteric ischemia and/or renal ischemia. In some embodiments, the stent treats the stenosis or restenosis by increasing endothelial cell proliferation and/or inhibiting smooth muscle cell migration. In some embodiments, the stent treats the ischemia by increasing tissue regeneration.

### 3. MATERIALS AND METHODS

Generation of MSC exosomes. Human MSCs were obtained from ATCC (PCS-500-012). Authentication and validation were performed to confirm the identity of the cells by flow cytometry characterization of common MSC surface markers. Exosomes were collected as previously described. In brief, MSCs were cultured until 80% confluence and washed by serum-free medium for three times. Then, the cells were incubated with serum free medium for another three days to allow exosome secretion. The conditioned media were then collected and filtered through 0.22 μm to remove residual cells and debris. Ultracentrifugation was then performed at 100,000 g for 2 hours to collect MSC-XOs (Beckman Coulter XL90 ultracentrifuge). In several experiments, fluorescently labeled exosomes were used. Purified MSC-XOs were mixed with 1 μM DiD or DiR (Invitrogen, Life Technologies) and incubated for 30 min at 4° C., then free dye was removed through centrifugal filter (10 KDa). MSC-XOs were washed three times with PBS.

Characterization of exosomes. The concentration of exosomes was examined with a NanoSight LM10 (Malvern Instruments Ltd., UK). The morphology of exosomes was visualized using a transmission electron microscope (TEM, JEOL JEM-2000FX). RNA sequencing and proteomics of MSC-XOs were performed as previously described. Briefly, exosomal RNA was isolated using a total exosome RNA isolation kit (Qiagen's exoRNeasy Serum Plasma Kit). Libraries were quantified by a Quant-iT™ dsDNA High Sensitivity Assay Kit (ThermoFisher) and sequenced on an Illumina NextSeq500 using a mid-output V2 kit. LC/MS/MS analysis of the exosome samples was performed on an Easy Nano ultra-high-pressure liquid chromatograph coupled to a Q Exactive HF-X Hybrid Quadrupole-Orbitrap mass spectrometer (ThermoFisher Scientific).

Fabrication of EES. All materials were purchased from Sigma Aldrich. All reagents were used as received. Stents were purchased from eSutures. Medtronic Integrity RX Coronary Stent System 2.5 mm×12 mm and Boston Scientific Rebel PtCr Monorail Coronary Stent was used in the modification experiments. EES made from that were used in animal studies for a head-to-head comparison. Medtronic Resolute Integrity RX Zotarolimus-Eluting Coronary Stent System (2.5 mm×12 mm) and Boston Scientific Ion Monorail Paclitaxel-Eluting Platinum Chromium Coronary Stent System was used as the DES control in the present studies. To fabricate EES, stents were first expanded and removed at a pressure of 4 atm by an angioplasty balloon. Then, stents were ultrasonically cleaned with acetone, ethanol, and deionized water. After drying, the stents were placed in mixed acid solution (1:1 (v/v) nitric acid-hydrofluoric acid) for 30 min at room temperature (RT). Following acid treatment, the stents were washed with deionized water for three times and placed in 10 N NaOH at 80° C. for 30 min. Stents were rinsed with deionized water for another three times. The hydroxylated stents were then immersed in a 5% (v/v) solution of (3 aminopropyl) triethoxysilane (APTES) in ethanol overnight and kept thermostatic at 60° C. under nitrogen flow. Following rinsing with deionized water and blow-dried under a stream of nitrogen, the modified stents were stored in individual containers for further usage. 4-Carboxyphenylboronic acid was activated using 1-Ethyl-3-(3-dimethylaminopropyl) carbodiimide (EDC) and N-hydroxysuccinimide (NHS) and reacted with amino groups on the stent. 1,2-distearoyl-sn-glycero-3-phosphoethanolamine-N-[amino(polyethylene glycol)-5000]-N-hydroxysuccinimide (DSPE-PEG₅₀₀₀-NHS) was reacted with 3-Amino-1,2-propanediol with a mole ratio of 1:3 to provide dihydroxyl groups. The obtained products were dialyzed against DI water (MWCO: 3 KDa) and lyophilized. Finally, the dihydroxyl-modified DSPE was added to the stents overnight to generate DSPE-conjugated stents. DSPE-modified stent was incubated with 10¹² exosomes in 4° C. overnight to fabricate the final product EES.

Physiochemical Characterization of EES. Time-of-flight secondary ion mass spectrometry (ToF-SIMS) analyses were conducted using a TOF SIMS V (ION TOF, Inc. Chestnut Ridge, NY) instrument equipped with a Biₙ^{m+} (n=1-5, m=1, 2) liquid metal ion gun, Cs⁺ sputtering gun and electron flood gun for charge compensation. X-ray Photoelectron Spectroscopy (XPS) analysis was conducted by SPECS XPS/UVS System with PHOIBOS 150 Analyzer (SPECS Surface Nano Analysis GmbH, Berlin, Germany). XPS data analysis was performed with the curve fitting program (CasaXPS, Version 2.3.17PR1.1). Morphology of stents was visualized via scanning electron microscopy (SEM) imaging. Samples were fixed with 2% glutaraldehyde, and then dehydrated in gradient ethanol successively for 10 min each, at last, dried in hexamethyldisilazane (Sigma-Aldrich) for imaging (JEOL 6010LA SEM, JEOL ltd, Japan).

Cell lines. Human primary umbilical vein endothelial cells (HUVEC, PCS-100-013), human primary aortic smooth muscle cells (PCS-100-012), and primary coronary artery endothelial cells (HCAEC, PCS-100-020) were purchased from ACTT. U937 human monocytes were purchased from Millipore Sigma.

Biological characterization of EES. For exosome release studies, one EES stent was incubated with 1 mL PBS in 37° C., and the solution was taken out at different time points for NanoSight quantification of exosome concentrations. For tube formation assay, HUVECs were seeded onto Matrigel (BD Biosciences)-coated plate wells, and a piece of BMS or EES was placed gently on the cells. Images were taken by an epi-fluorescent microscope (Olympus, Tokyo, Japan). Tube nodes were quantified by ImageJ (NIH). For in vitro endothelialization assay, HUVECs were seeded on glass slides and allowed to be confluent. A piece of BMS or EES was placed gently on top of the HUVEC monolayer. Four days later, stents were gently removed, washed with PBS and fixed with glutaraldehyde for SEM imaging.

Cell proliferation assay. Cells were seeded in 96 well plates overnight and incubated with a piece of BMS or EES for additional two days. Then, BMS or EES were removed gently and Cell Counting Kit-8 (CCK-8, Sigma) reagent was added. Cell viability was determined by measuring absorbance wavelength of 450 nm and reference wavelength of 630 nm with a VersaMax™ Microplate Reader.

Smooth muscle cell migration assay. HUVECs were seeded in 24-well plates. A piece of BMS or EES was gently placed on HUVECs. Trans-well chambers with a 8-μm pore size were placed. SMCs were seeded on the trans-well chambers. The plates were incubated for 6 h then the chambers were removed and fixed with 4% formaldehyde. The non-migrated SMCs were removed with cotton swap gently. The migrated SMCs on the back side of the filter were stained with 0.05% crystal violet and viewed via a microscope (Olympus, Tokyo, Japan).

In vitro thrombosis and adhesion assay. Fresh platelet rich plasma (PRP) was collected from whole blood of healthy Sprague Dawley rats (male, 8 weeks). BMS or EES were co-cultured with PRP for 30 min at 37° C. Then, BMS or EES were washed with PBS and fixed in 2.5% Glutaraldehyde for SEM imaging. FITC-CD42b (eBioscience) staining was used to confirm the platelets on stent surface. BMS or EES were incubated with U937 monocytes (1×10⁶ cells/mL) and TNF-α (10 ng/mL) under rotation for 4 hours to stimulate in vivo conditions. After that the stents were washed with PBS and fixed in 2.5% Glutaraldehyde for further SEM imaging. FITC-CD11c (eBioscience) staining was performed to confirm the monocytes on stent surface.

Antibodies. Primary antibodies used in the present disclosure: Anti-Von Willebrand Factor antibody (ab6994) (vWF, ABCAM), Anti-alpha smooth muscle Actin antibody (ab7817) (α-SMA, ABCAM), Anti-Ki67 antibody (ab15580) (ABCAM), Rhodamine labeled Lens Culinaris Agglutinin (LCA, VECTOR LABORATORIES, INC.), CD81 antibody (sc-166029, Santa Cruz Biotechnology), Anti-ALIX antibody (ab186429, ABCAM), TSG101 antibody (NB200-112, NOVUS Biologicals), Anti-CD68 antibody (ab955) (ABCAM), Anti-Dystrophin (ab15277, ABCAM), Anti-Glucose Transporter GLUT1 (ab40084, ABCAM), Anti-Mannose Receptor (ab64693, ABCAM), Anti-MHC Class II (ab23990, ABCAM), Anti-CD31 (ab119339, ABCAM) Anti-VE cadherin (ab7047, ABCAM). Secondary antibodies used in the present disclosure: Goat Anti-Rabbit IgG H&L (Alexa Fluor® 488) (ab150077), Goat Anti-Mouse IgG H&L (Alexa Fluor® 488) (ab150113), Goat Anti-Mouse IgG H&L (Alexa Fluor® 594) (ab150116).

Immunocytochemistry. Cells were rinsed once with PBS and fixed in 4% paraformaldehyde (PFA) (Electron Microscopy Sciences; 15710) for 20 min, followed by permeabilization and blocking with Dako Protein blocking solution (DAKO; X0909) containing 0.1% saponin (Sigma-Aldrich; 47036) for 1 h at room temperature to prevent non-specific binding. Cells were incubated in primary antibodies overnight at 4° C. and then in secondary antibodies for 1.5 h at room temperature. ProLong™ Gold Antifade Mountant with DAPI (ThermoFisher) was used to counter-stain nuclei and slow the fade of fluorophore. All antibodies and kits were used according to instructions from the vendors. Cells were examined with a confocal microscope (Zeiss LSM 710).

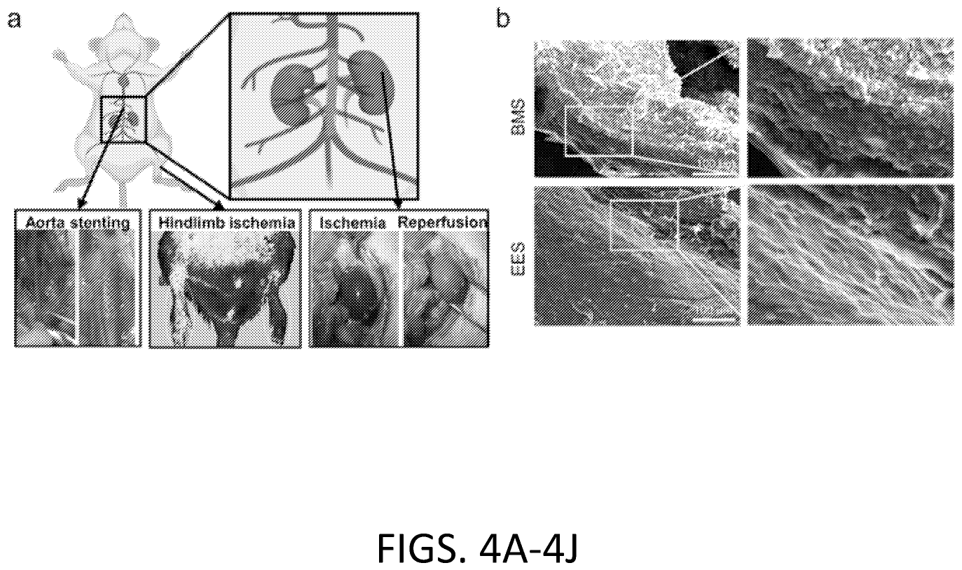

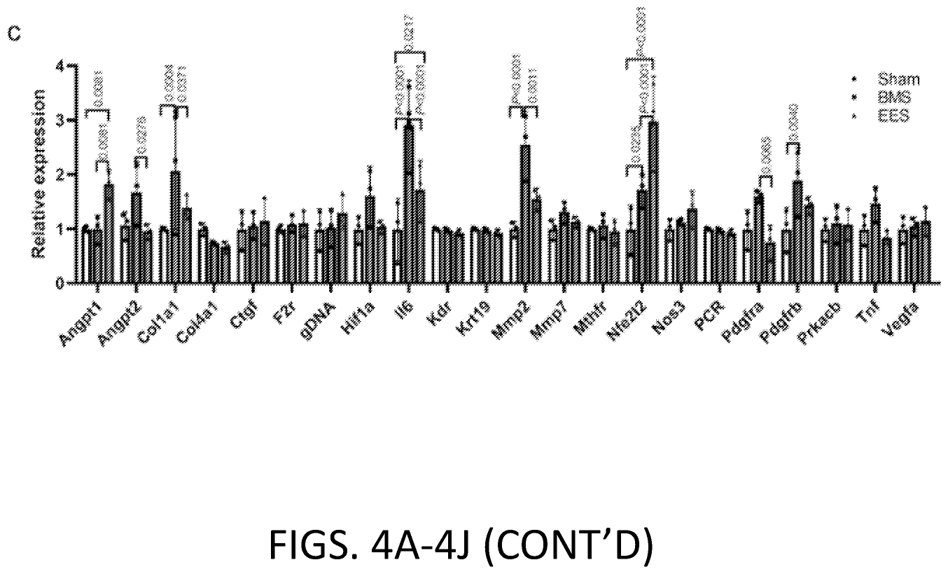

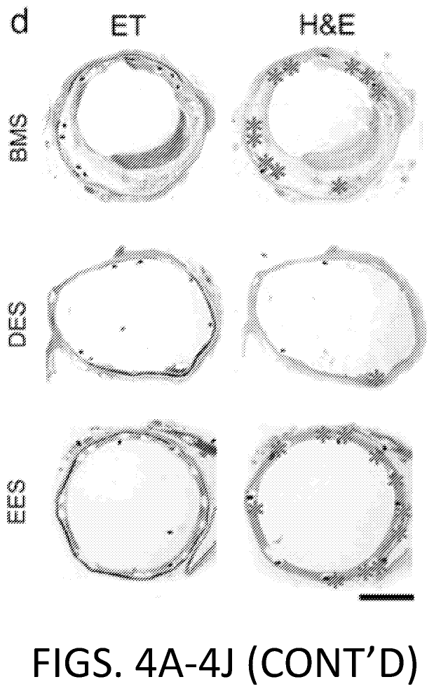

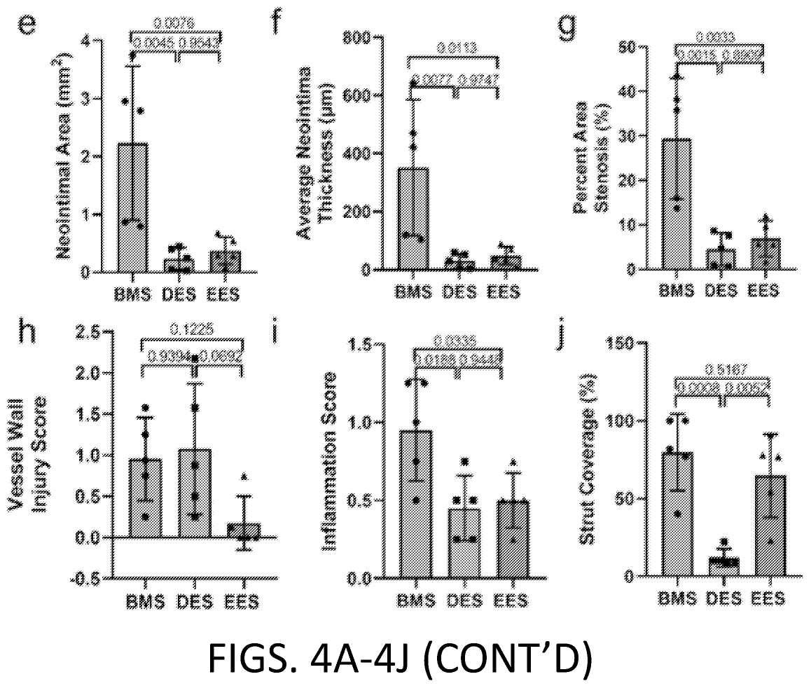

Sprague Dawley rat studies. All experimental protocols were approved by the Institutional Animal Care and Use Committee at North Carolina State University. Sprague Dawley (SD) rats were purchased from Charles River Laboratories. The use of the rat abdominal artery to test ISR due to stenting has been supported by the literature for decades. SD rats (300-500 g, male) were anesthetized with 2% isoflurane in oxygen. After median laparotomy, the abdominal aorta was isolated to give enough length for stents deployment (3 cm). Then, two vascular clips were placed to create an aorta segment. A small incision was created at one end. This segment was then flushed with heparin carefully. A balloon catheter was inserted through the small incision and inflated to deploy a stent into the aortic segment. After that, the balloon was removed, and the small incision was closed with a 9-0 suture. Finally, the vascular clips were removed. Intravenous heparin (100 U/kg) was administered after stent deployment. The abdomen was closed with 4-0 sutures and the animals were allowed for recovery. Healthy SD rats were randomized into three groups (n=6): Sham, BMS, EES. Stents were collected on day 7 and used for PCR arrays (FIG. 4B, n=3), SEM (FIG. 4C, n=3), or immunofluorescent staining (FIG. 5, n=6).

Renal ischemia-reperfusion injury model. Bi-lateral renal ischemia-reperfusion (RIR) injury model was performed as previously described with slight modifications. Briefly, bilateral renal pedicle occlusions were induced by vascular clamping of 45 min. Ischemia was confirmed by the color change of the kidney. With the removal of the clamp, a color change in kidney indicated proper reperfusion. Stenting was performed during the ischemia surgery. The animals were randomized into four groups (n=5): Sham, RIR, RIR+BMS stenting, and RIR+EES. On day 1, 7 and 28, blood samples (1 mL) were collected to test kidney functional biomarkers BUN (blood urea nitrogen, Urea Nitrogen Colorimetric Detection Kit, EIABUN, ThermoFisher) and Cre (Creatinine Assay Kit (ab65340), AMCAM). After euthanasia, kidneys were harvested and bisected longitudinally. Kidney tubular necrosis, inflammatory cells, and vacuolar degeneration were analyzed with H&E staining. Fibrosis was assessed by Trichrome staining. In Situ Cell Death Detection Kit, Fluorescein (TUNEL, Sigma) was used to detect apoptotic cells. BMS and EES were collected from euthanized animals on day 28 after treatment for histological analysis (HE and ET staining). A group of animals (RIR injury model) were added for DES stenting and histology (n=5, FIGS. 4D-4J).

ApoE^{-/-} rat studies and hind limb ischemia model. ApoE^{-/-} rats (8-week, male) were purchased from Horizon Discovery and fed with a high fat diet during the entire study. Hindlimb ischemia was induced on the right limb before stent deployment. Briefly, rats were anesthetized with 2% isoflurane in oxygen and the surgical area were shaved. The targeted vessels were visualized by creating an incision overlying the proximal, medial portion of the right hindlimb. The femoral artery and vein were dissected away from the femoral nerve and ligated to target the proximal removal of a 1 cm segment. The entry incision through the skin was then closed with a suture (5-0 silk). Blood flow in both hindlimbs was assessed using a Laser Doppler perfusion imager (PeriCam PSI NR). The rats were randomly divided into three groups (n=12): BMS, DES and EES to receive stent deployment in their abdominal aortas as previously described. A subgroup of rats (n=6) was sacrificed 7 days later. The aortas were cut into two parts: one for PCR arrays (FIGS. 6C-6D, n=4) and the other for immunofluorescent staining (FIGS. 6A-6B, FIGS. 6E-6F, n=5). All other animals were followed until day 28.

Histopathological analysis. The harvested stented aortas were washed with PBS for three times and fixed with 4% PFA. After that, the aortas were embedded in methylmethacrylate and sent for sectioned. Sections with 5-μm thickness were cut from the proximal and middle parts of the stented aorta and mounted on 3-Aminopropyltriethoxysilane (APES) coated slides. After that the slides were de-plastified and stained with Hematoxylin & Eosin (H&E) and Elastin Trichrome (ET). Immunostaining was performed on tissues (organs, limbs, or stented aortas). Tissue sections were fixed with 4% PFA for 20 min and then blocked with Dako Protein Blocking solution containing 0.10% saponin for 1 h. After that slides were incubated with primary antibodies overnight at 4° C. and then with secondary antibodies for 1.5 h at room temperature. ProLong™ Gold Antifade Mountant with DAPI was used. The slides were imaged with a confocal microscope (Zeiss LSM 710). Elastin trichrome staining was used for histomorphometry analysis using Image-Pro Plus 7 software. All cross-sections were measured, and the following morphometric data were collected: external elastic lamina (EEL) area (mm²), internal elastic lamina (IEL) area (mm²), and lumen area (mm²). From those direct measurements, all other histomorphometric parameters were calculated, such as neointimal area (mm²): IEL area minus lumen area; percent area stenosis based on IEL area (%): 100×neointimal area/IEL area; Average neointima thickness (μm): 1000×(√(IEL area/π)−√(lumen area/π)). Samples were divided into four section and quadrant injury was scored. Device biocompatibility was assessed based on H&E staining and quadrant inflammation was scored. Semi-quantitative scoring was as follows: 0, not present; 1, present, but minimal feature; 2, notable feature, mild; 3, prominent feature that does not disrupt tissue architecture, moderate; 4, overwhelming feature, severe.

Dihydroethidium staining. The aortas were harvested, and the stents were carefully removed. Cryosections were prepared for dihydroethidium (DHE) staining. Briefly, slides were rinsed once in pure water to wash out OCT compound, then placed in DHE staining solution (5 μM) immediately. After 20 min incubation at room temperature, the slides were immersed in pure water for washing (1 min, three times). The slides were imaged with a confocal microscope (Zeiss LSM 710).

ROS detection. OxiSelect™ In Vitro ROS/RNS Assay Kit (Green Fluorescence) was used for ROS detection. 2 mg aorta in 100 μL PBS was homogenized on ice. Then, spin at 10,000 g for 5 min to remove any tissue debris. The homogenate was assayed. The plate was read with a fluorescence plate reader at 480 nm excitation/530 nm emission (Fluorescence Spectroscopy, Perkin Elmer Model LS-3B).

PCR array. Total RNA was isolated from the stented arteries using the RNeasy mini kit (QIAGEN, Hilden, Germany). Complementary DNA (cDNA) was synthesized using iScript™ cDNA Synthesis Kit (Bio-Rad). Rat thrombosis-related qPCR array was purchased from Bio-Rad and the data was analyzed using the Bio-Rad qPCR analysis software. GeneQuery rat macrophage polarization markers qPCR array kit was purchased from ScienCell. SsoAdvanced™ Universal SYBR® Green Supermix (Bio-Rad) was used to perform the qPCR studies. The plates were read on Roche LightCycler® 480. Data has been submitted to NCBI (GSE155793).

Statistics. All quantitative experiments were done in triplicate unless otherwise indicated. Data were shown as mean±S.D. GraphPad Prism was used for statistical analysis. Student's two-tailed independent t-test was used to determine differences between two groups. Comparison of more than two groups were performed using one-way analysis of variance (ANOVA) followed by Tukey's multiple comparisons. Grouped data were analyzed using two-way ANOVA followed by Tukey's multiple comparisons. P<0.05 was considered statistically significant.

### 4. EXAMPLES

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

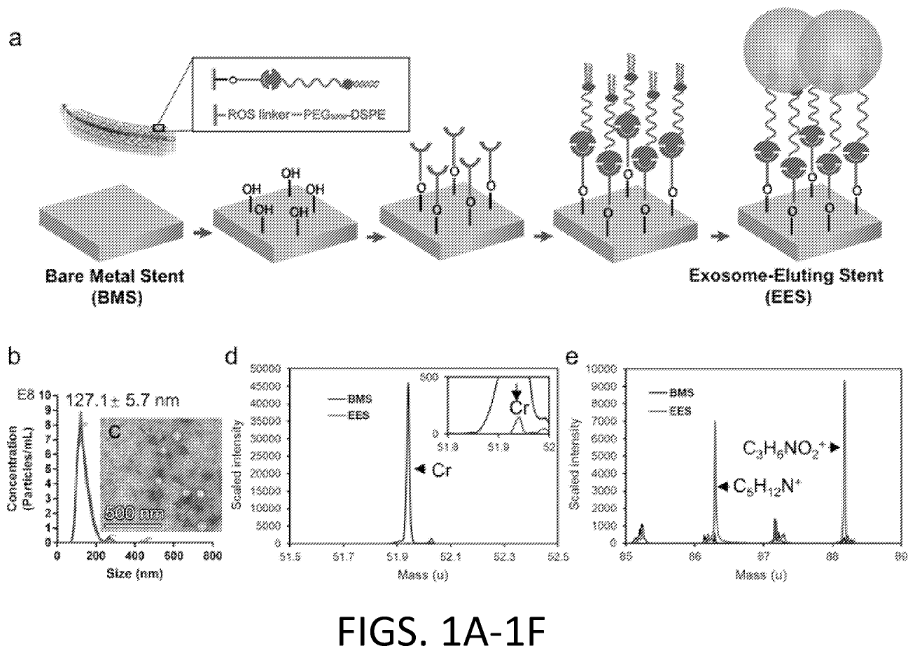

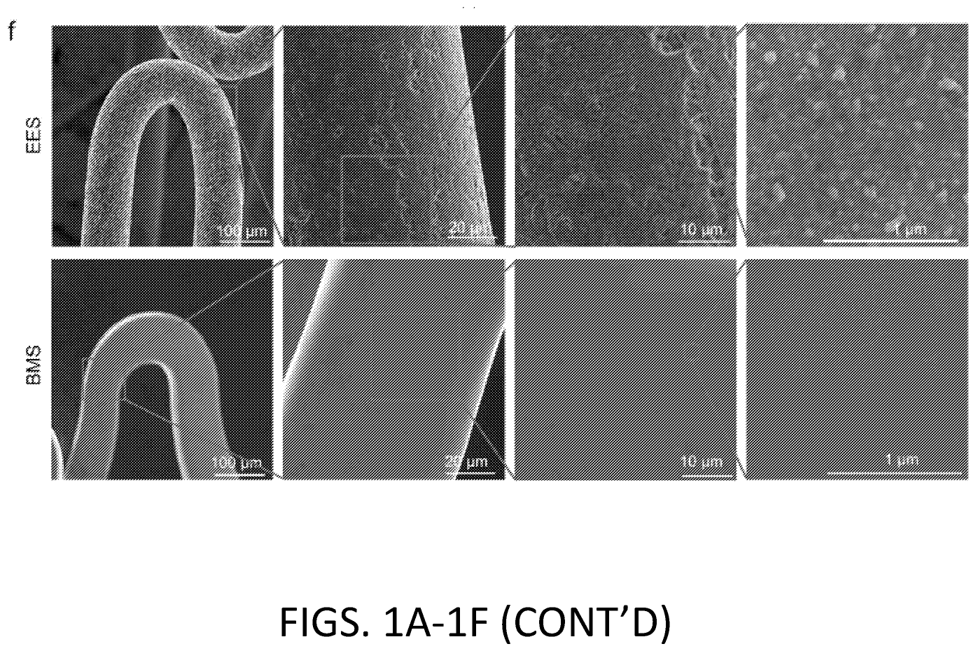

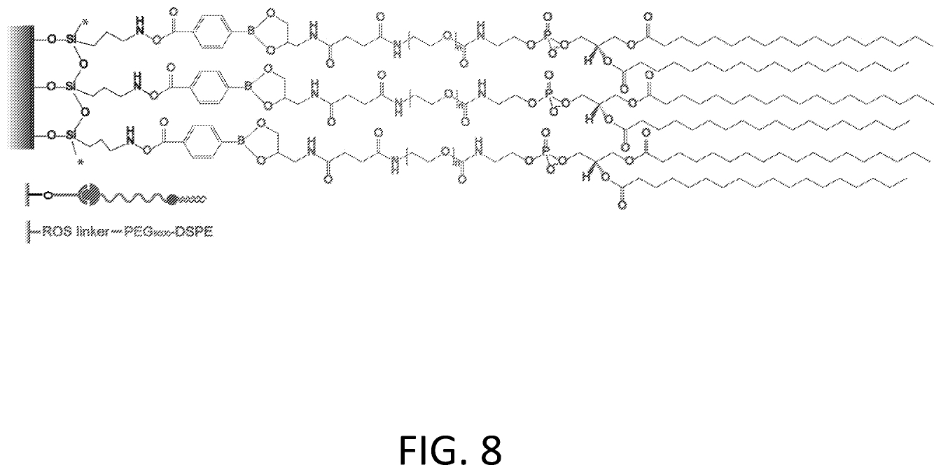

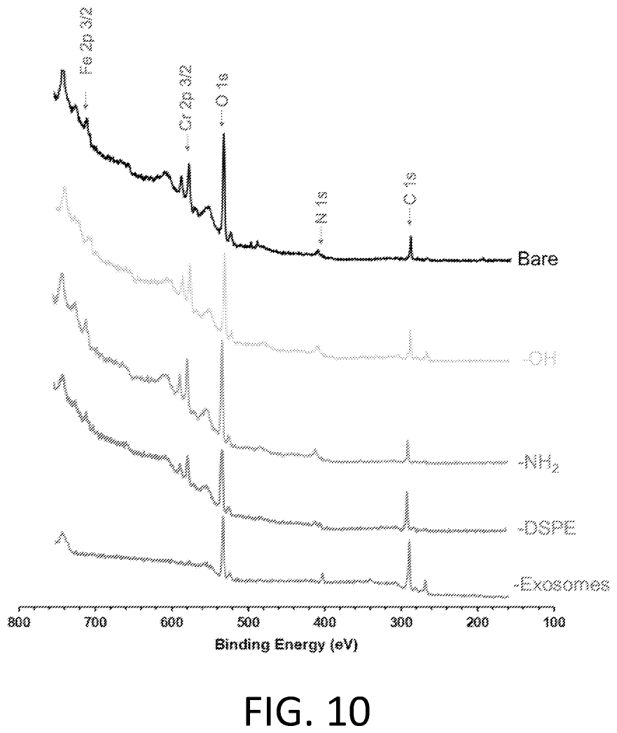

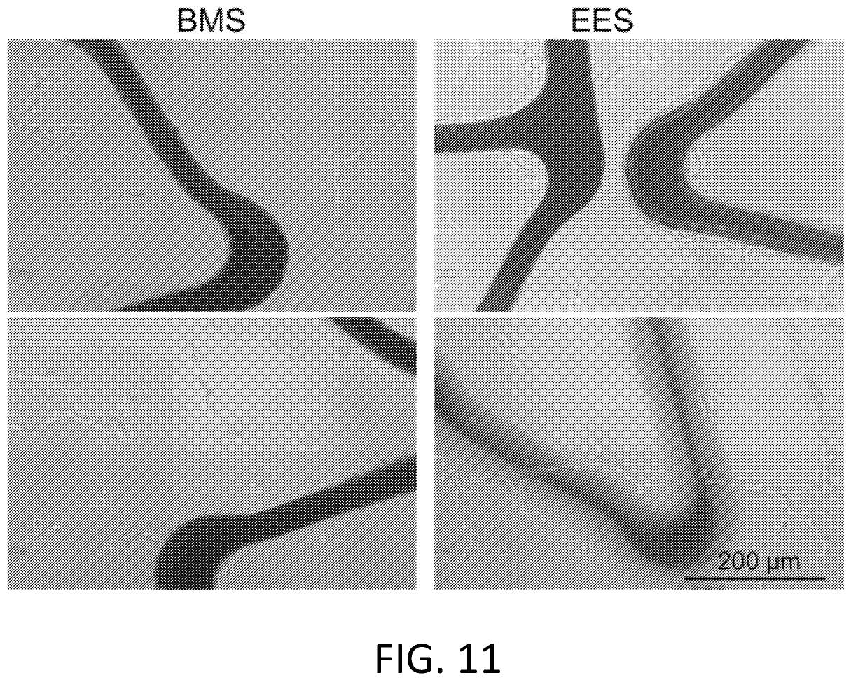

Fabrication and characterization of exosome-eluting stents. As shown in FIG. 1A and FIG. 8, 1,2-distearoyl-sn-glycero-3-phosphoethanolamine (DSPE)-conjugated stents were prepared first and then integrated with MSC-XOs with an ROS linker. To coat MSC-XOs onto the stent, the surface of stents was first modified by 3-aminopropyltriethoxysilane (APTES) to generate amines groups for further modification. Silanization with APTES was performed after surface hydroxylation. Then, 4-Carboxyphenylboronic acid was reacted with amino groups on the stent. DSPE-PEG₅₀₀₀-NHS was reacted with 3-Amino-1,2-propanediol to provide the dihydroxyl groups. The dihydroxyl modified DSPE was then added to the above stent overnight to generate DSPE coated stents. After that, the DSPE-modified stent was incubated with 10¹² exosomes in 4° C. overnight to fabricate an EES. MSC-XOs were collected and characterized as previously described. The size at the peak concentration was 127.1 nm (FIG. 1B). TEM image confirmed the morphology of MSC-XOs (FIG. 1C). Western blot further proved the expressions of common exosomal surface markers such as Alix, TSG101 and CD81 (FIG. 1C). MicroRNA sequencing revealed the cargo miRNAs in MSC-XOs (FIG. 9B), including those have been reported in MSC-XO mediated tissue repair, such as miR-23 and the let-7 family to recipient cells to promote angiogenesis and cell survival. Furthermore, proteomics studies of MSC-XOs identified a variety of proteins including fibronectin, collagen alpha-1, and thrombospondin-2 (FIG. 9C). EES was characterized by X-ray photoelectron spectroscopy (XPS, FIG. 10) and time-of-flight secondary ion mass spectrometry (ToF-SIMS, FIGS. 1A-1D). XPS demonstrated the binding energy change of Fe 2p 3/2 during surface hydroxylation and silanization. The area changes of N Is, C is and O is indicated the conjugation of DSPE and MSC-XOs. To further confirm the efficiency of coating, ToF-SIMS spectrometry was employed to examine the molecular compositions of EES by direct chemical detection of exosome membranes. The lack of Cr signal (m/z 52) from BMS indicated an exosome coverage (FIG. 1D). Membrane phospholipids of exosomes showed signals at C₃H₆NO₂⁺ (m/z 88) from the amino acid serine, which was present in phosphatidylserine, and C₅H₁₂N⁺ (m/z 86) from phosphocholine, which was present in phosphatidylcholine (FIG. 1E). Those signals confirmed the effective coating of MSC-XOs on the stent. SEM imaging showed a smooth surface of BMS, while a nanoscale roughness surface of EES due to exosome coating (FIG. 1F). The exosome-based nanoscale roughness could accelerate endothelial cell adhesion and proliferation and inhibit the adhesion and activation of platelets.

### Example 2

In vitro exosome release from EES and blood compatibility. To release MSC-XOs into the blood stream and peripheral ischemic tissue timely, benzeneboronic acid pinacol ester group was used here to react with local ROS stimuli (FIG. 2A). MSC-XOs slowly released into the physiological environment without stimulation, and around 20% exosomes were released from EES after 48 hours. With the addition of H₂O₂, around 40%˜60% exosomes were gradually released (FIG. 2B) in 48 hours.

To confirm the bioactivity of exosomes after eluting, released exosomes and EES were further characterized. The size of released exosomes didn't change (FIG. 2C). FIG. 2d showed the morphology of EES after 2 days of release studies in PBS and 0.1 mM H₂O₂ respectively. To verify the biocompatibility of EES, a small piece of EES (with red fluorescent DiD-labeled MSC-XOs) was co-cultured with human umbilical vein endothelial cells (HUVECs) for 12 hours on Matrigel (FIG. 2E). HUVEC tube formation and uptake of the released MSC-XOs is evident (FIG. 2E). In addition, blood compatibility of EES was evaluated in vitro by incubating BMS or EES with platelet-rich plasma (PRP) and inflammatory cells. Compared to BMS, EES significantly reduced the adhesion of both activated platelets and TNF-α activated U937 monocytes (FIGS. 2F-2H).

### Example 3

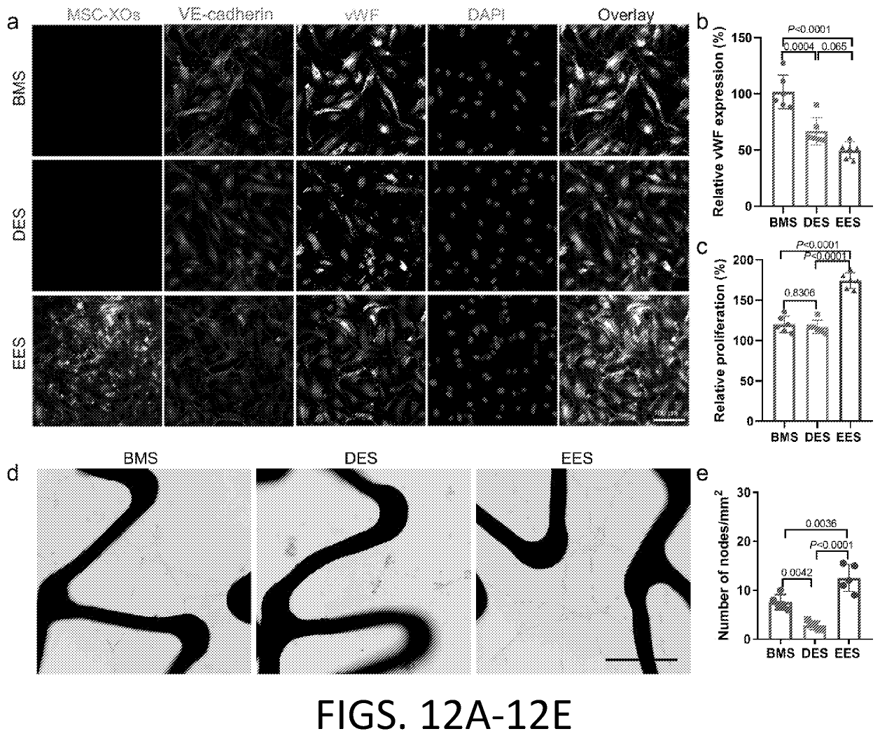

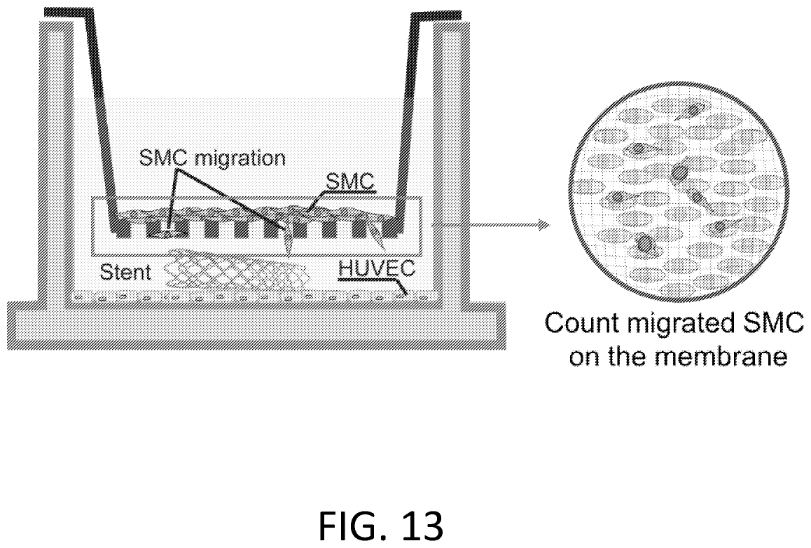

EES promotes endothelial cell proliferation and inhibits smooth muscle cell migration. Released exosomes are likely to interact with endothelial cells (ECs), SMCs, and the adjacent injured tissue (FIG. 3A). Four hours of co-culture with EES promoted endothelial tube formation (FIG. 3B), with an increase in numbers of endothelial network nodes (FIG. 3C) as well as longer tubes. Also, HUVEC adhesion to EES was evident (FIG. 11). MSC-XOs reduced the expression of von Willebrand Factor (vWF) in HUVEC (FIGS. 3D-3E). It has been reported that down regulation of vWF promotes the proliferation and migration of endothelial cells. CCK-8 assay further verified the enhanced proliferation of HUVEC with EES (FIG. 3F). To examine endothelialization in vitro, stents were cultured with confluent HUVEC for additional 4 days and imaged by scanning electron micrograph (SEM). SEM revealed the remarkable cell coverage on EES, but not on BMS, by HUVEC (FIGS. 3G-3H). Similar effects of EES were also observed on human primary coronary artery endothelial cells (HCAECs, FIG. 12). Collectively, MSC-XOs eluted from EES encouraged the proliferation and tubing of both HUVECs and HCAECs. Furthermore, EES inhibited the trans-well migration capacity of SMCs (FIG. 13; FIGS. 3H-3J). An up-regulation of α-SMA expression was also observed in EES co-cultured SMCs (FIGS. 3K-3L) compared to BMS, indicating a phenotypic modulation effects from the released exosomes. There was no significant change in SMC proliferation (FIG. 3M). Taken together, those in vitro experiments indicated that EES promotes endothelial tube formation and proliferation, but not the migration of SMC.

### Example 4

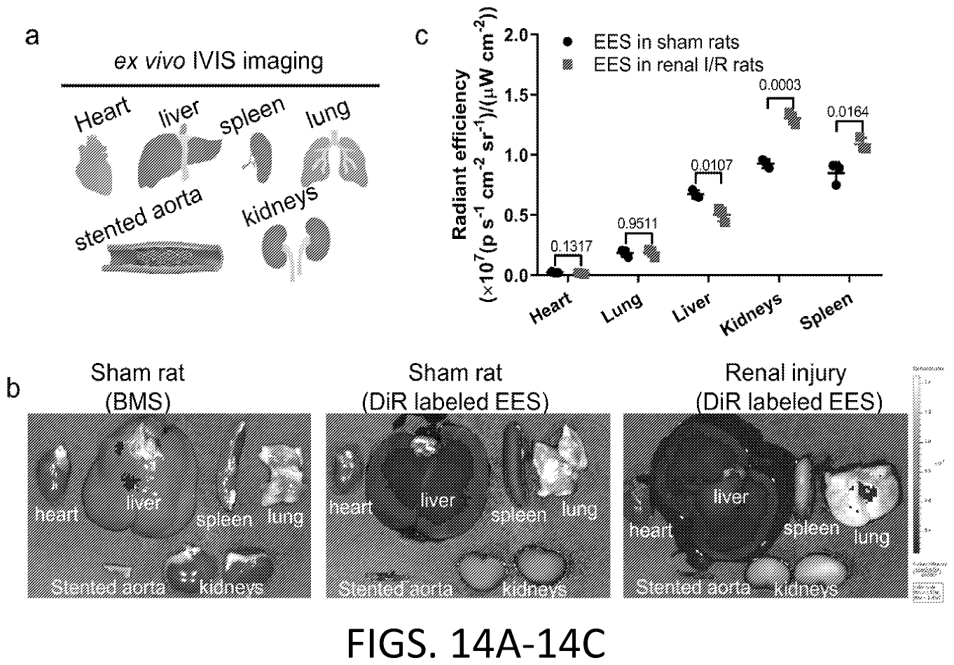

Abdominal aorta stenting in rats with renal ischemia-reperfusion. As shown in FIG. 4A, a rat model of bilateral renal ischemia-reperfusion injury and a rat model of unilateral hindlimb ischemia were employed to examine the biocompatibility and therapeutic benefits of EES during stenting. Briefly, the abdominal aorta was separated from surrounding tissue and placed with a commercially-available BMS or an EES (made from the same BMS). Three days after stenting, a group of rats were euthanized and major organs, including the heart, liver, spleen, lungs, and kidneys were extracted for ex vivo imaging using the IVIS system (FIG. 14). The distribution of MSC-XOs in the spleen and the kidneys was significantly higher in rats with renal ischemia-reperfusion injury compared to sham-operated rats.

Seven days after stenting, aortas with BMS or EES were harvested for SEM imaging (FIG. 4B). The BMS was full of irregular adhesions, while EES exhibited a relative smooth surface. Further, thrombosis related PCR array was performed to investigate gene expressions in the local blood vessel tissues 7 days after stenting (FIG. 4C). The results indicated that EES stenting reduced the expressions of Angpt2, Col1a1, Hif1a, Il-6, Mmp2, Pdgfra and Pdgfrab, while enhancing the expressions of Angpt1 and Nfe2l2, as compared to BMS stenting. Angpt1 positively regulates vascular remodeling. The reduced expressions of Il-6 and Mmp2 demonstrated that EES ameliorates inflammation and fibrosis in the local environment. On the other hand, Nfe2l2 drives the expression of cytoprotective genes in response to oxygen stress.

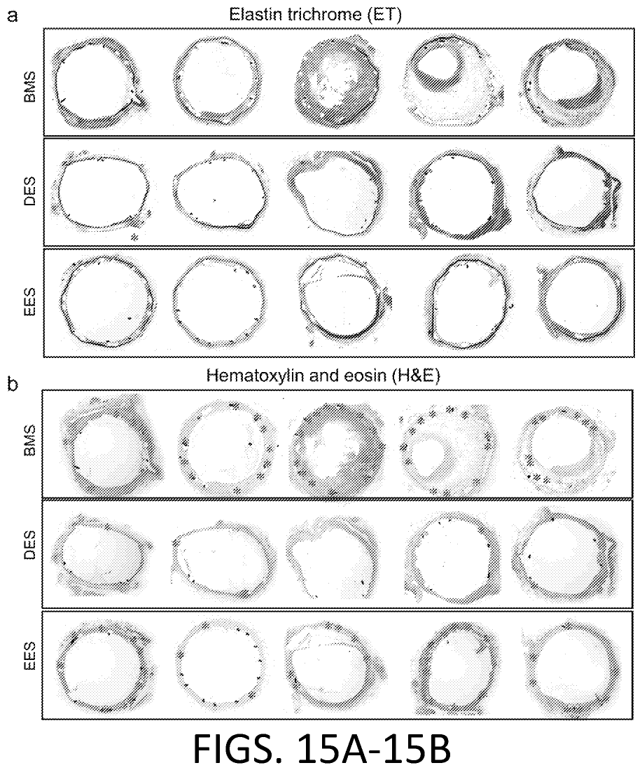

A DES group was added as another control for clinical relevance. The aortas were collected and sectioned on day 28, and then stained with hematoxylin & eosin (H&E) or elastin trichrome (ET). The histopathologic measurements are summarized in Table 1 (below). There were decreased lumen area and increased neointimal area in the BMS-treated animals, suggesting in-stent stenosis. The BMS group had a significantly thicker neointima and more severe lumen restenosis on day 28 than did the other two groups. The EES and DES groups had similar neointimal area and lumen area. Importantly, EES outperformed DES in terms of strut coverage. DES produce a dramatic reduction in stenosis. However, due to the fatal property of antiproliferation drugs on DES, strut coverage in DES (red asterisks, FIG. 4D and FIG. 15) is quite low. It has been reported that incomplete strut coverage was considered as a main reason of late in-stent restenosis in DES-implanted patients. Neointimal area, average neointima thickness, percent area stenosis, vessel wall injury score, inflammation score and strut coverage of BMS, DES and EES groups were quantified and shown in FIGS. 4E-4J.

TABLE 1
Histopathologic measurements on Day 28.

| | BMS | | DES | | EES | |
|---|---|---|---|---|---|---|
| Group | Mean | St. Dev. | Mean | St. Dev. | Mean | St. Dev. |
| EEL (mm²) | 5.14 | 0.0801 | 5.19 | 0.0763 | 5.24 | 0.0549 |
| IEL (mm²) | 4.84 | 0.170 | 4.84 | 0.0892 | 4.91 | 0.125 |
| Lumen area (mm²) | 2.61 | 1.14 | 4.61 | 0.244 | 4.53 | 0.196 |
| Neointimal area (mm²) | 2.22 | 1.18 | 0.232 | 0.171 | 0.377 | 0.209 |
| Average Neointimal | 351 | 208 | 30.5 | 22.7 | 49.1 | 27.4 |
| thickness (μm) | | | | | | |
| Stenosis (%) | 29.3 | 12.1 | 4.50 | 3.30 | 6.96 | 3.58 |
| Injury score | 0.952 | 0.449 | 1.07 | 0.708 | 0.200 | 0.291 |
| Inflammation score | 0.950 | 0.291 | 0.450 | 0.187 | 0.500 | 0.158 |

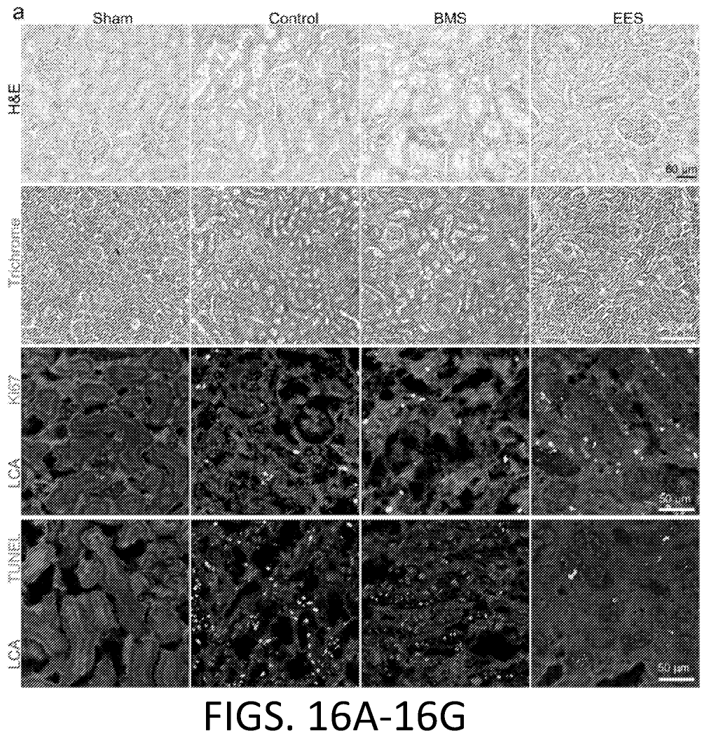

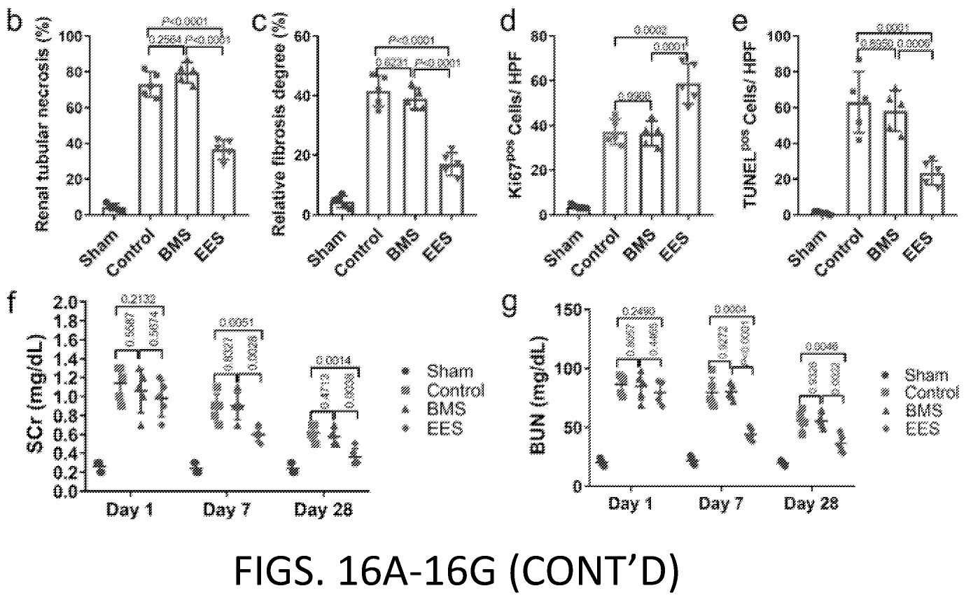

The reparative effects of EES stenting were evaluated in rats with renal ischemia-reperfusion injury (FIG. 16). H&E staining of the outer medulla of each kidney revealed that kidney tubule necrosis and inflammatory cells were significantly reduced in the EES-treated group (FIG. 16A). Severe tubular necrosis, including the cell necrosis and tubular dilation were observed in BMS group, while EES significantly ameliorate tubular necrosis (FIG. 16B). Trichrome staining indicated severe fibrosis after ischemia-reperfusion injury. EES treatment significantly ameliorate the fibrosis process (FIG. 16C). EES promoted endogenous repair as indicated by a higher percentage of proliferative (Ki67-positive) cells (FIG. 16D). Moreover, TUNEL staining revealed that EES treatment significantly decreased kidney tubules apoptosis (FIG. 16E). In addition, kidney functions of EES-treated animals were improved remarkably as compared to the ones treated with control BMS (FIG. 16F-16G).

### Example 5

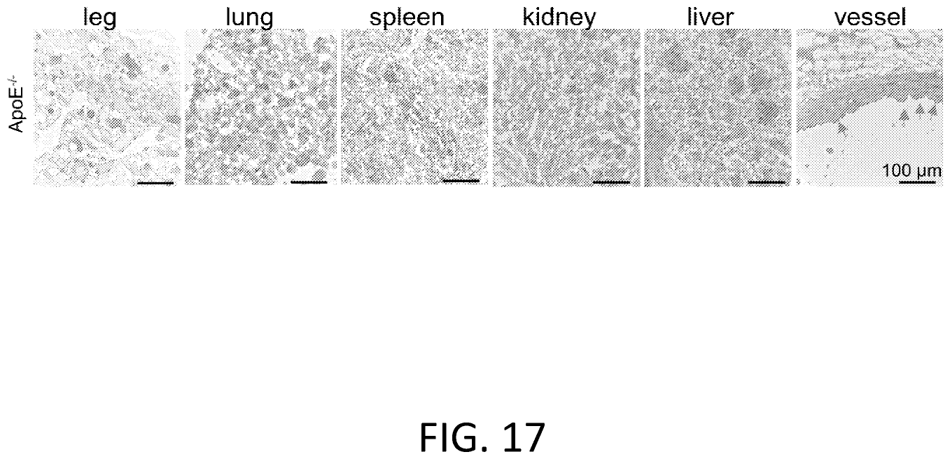

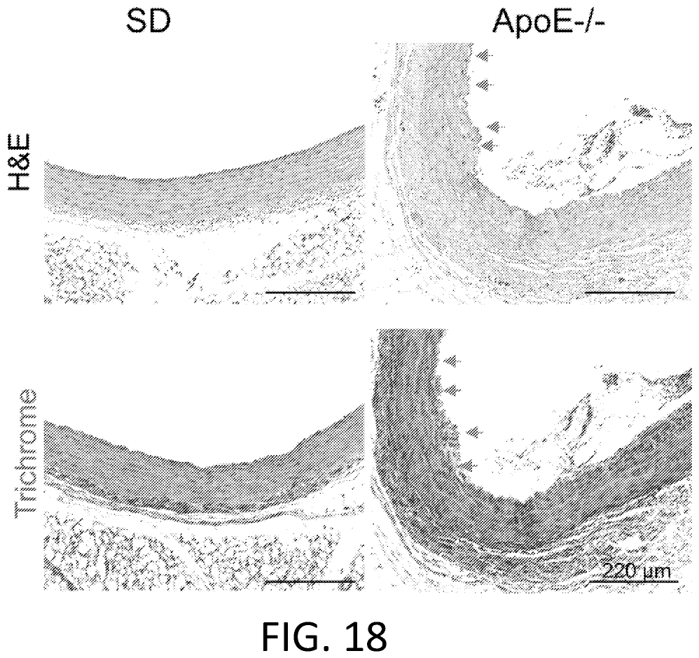

Abdominal aorta stenting in ApoE^{-/-} atherosclerotic rats. ApoE knockout (ApoE^{-/-}) rodents have been widely used as an atherosclerosis model and have provided valuable insights into the mechanisms underlying this disease. Consistent with published literature, ApoE^{-/-} rats exhibited increased lipid cores (FIG. 17) and lesions on the aortic vessel walls (FIG. 18). To better understand the vascular healing process, the effects of different types of stents on the proliferation/migration of SMC and EC were studied during neointimal formation in ApoE^{-/-} rats. As shown in FIGS. 5A-5B, the expression of α-SMA was uniform in both the media and the intima of the EES group, however, the expression of α-SMA in the BMS group and the DES group were irregular, with increased thickness of the vessel wall. SMCs with a reduced expression of α-SMA hold a higher potential of proliferation and migration. Glucose transporter-1 (GluT1) has been used as the marker of proliferating immature endothelial cells. GluT1 was used to evaluate intimal neovascularization. As shown in FIGS. 5E-5C, intimal neovascularization was reduced in the EES group and the distribution of CluT1 positive (GluT1⁺) cells was closer to the edge of the intima, demonstrating the incidence of intimal neovascularization (small vessels in the intimal area and around the strut area), highly related to late stent complications, was reduced in EES. CD31 was also used to stain functional endothelial cells (FIGS. 5F-5G), and there was no obvious difference among groups on day 7 after stent deployment. Neointimal microvessels can lead to fragile premature vessels, which are unstable and rupture-prone. These results indicate a higher expression of GluT1 and relatively low expression of CD31 in DES-treated vessels as compared to EES-treated ones. It is possible that antiproliferation drugs eluted from DES inhibited the growth of both smooth muscle cells and endothelial cells but could not prevent the formation of intimal microvessels. MSC-XOs may promote the maturation and function of endothelial cells, favoring the formation of a healthy neointimal layer.

MSC-XOs play a role in macrophage polarization to promote wound healing and modulate inflammatory milieu in atherosclerosis. On day 7, stented abdominal aortas were harvested. Both dihydroethidium (DHE) staining (FIG. 6A) and ROS assay (FIG. 6B) showed an overall enhanced ROS level of aortas after stent deployment. EES reduced the ROS level as compared to the BMS and DES groups. To reveal the immune-regulatory effects of EES, macrophage polarization-related PCR array was performed (FIGS. 6C-6D) to elucidate the gene expression patterns in the vascular tissue. The upregulation of FABP4 gene in the BMS group favors atherosclerosis. Compared to DES, EES treatment further reduced the expressions of inflammatory mediator chemokines such as (C—C motif) ligand 2 (CCL2), IL-1β and IL1R1, which are reportedly associated with inflammation and adverse vascular remodeling. Compared to both BMS and DES groups, EES treatment led to a higher expression in M2 macrophage markers, MRC-1 and CD163, and a higher expression of anti-inflammatory cytokine, IL10. EES also favors the M2 polarization by showing a higher expression of CD163 and MRC1. Immunohistochemistry (FIGS. 6E-6F) further demonstrated a decrease in total macrophage numbers and an increased ratio of M2 macrophages (CD206⁺/CD68⁺ ratio) in the EES-treated group.

### Example 6

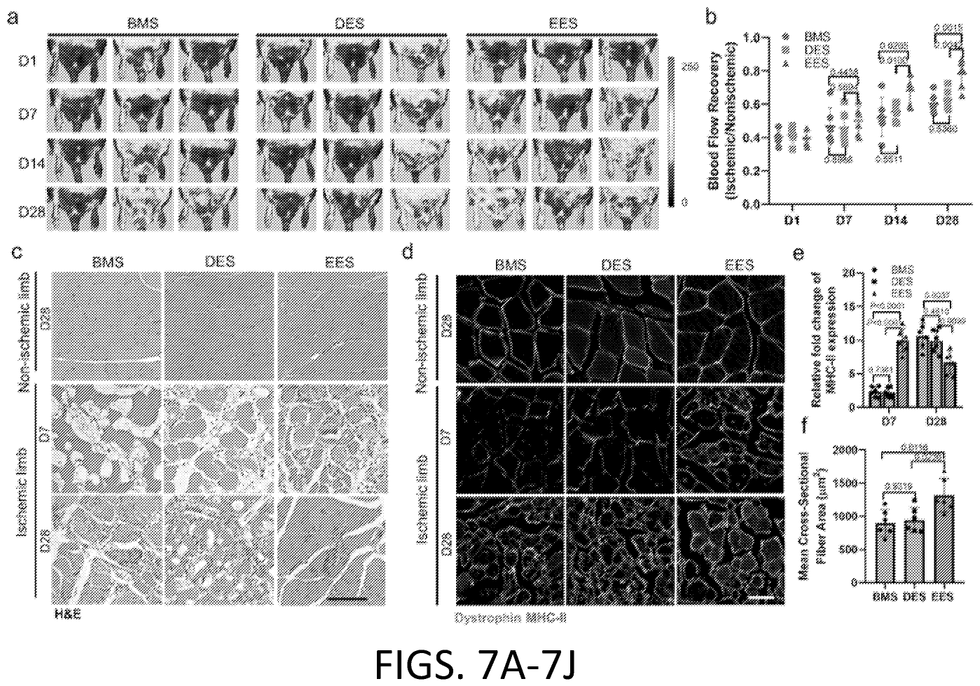

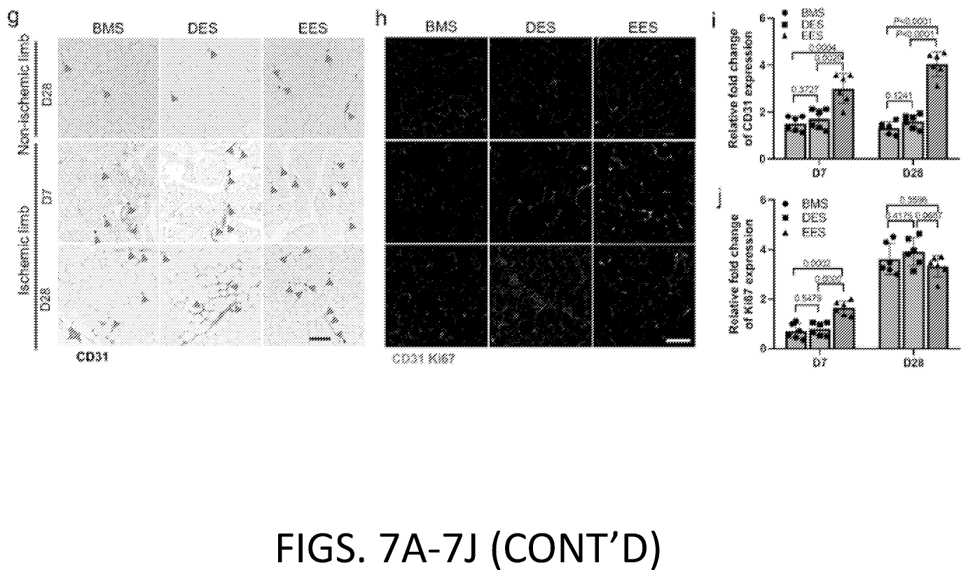

Treatment effects of EES in ApoE^{-/-} rats with hindlimb ischemia. To demonstrate the therapeutic benefit of EES on peripheral arterial disease, BMS, DES and EES were deployed in ApoE^{-/-} rats with unilateral hindlimb ischemia injury. Laser Doppler perfusion imaging demonstrated the therapeutic efficacy of EES in restoring blood flow (FIGS. 7A-7B). H&E staining showed that muscle bundles were severely impaired in both BMS and DES groups, while EES treatment led to a preservation/repair of healthy muscle morphology (FIG. 7C). Co-stained was performed for MHC II and Dystrophin. MHC-II is mainly expressed by CD4⁺ T cells. As shown in FIGS. 7D-7F, EES favored a quick immune response and myofiber repair process. Dystrophin positive fibers were significantly higher than BMS and DES groups. Measurement of fiber cross-sectional area indicated a decrease in myofiber sizes on the ischemic sides compared to non-ischemic limbs in all groups. EES treatment lead to intact and regular morphology as compared to the other two stent groups, indicating either a protective or reparative effect from EES treatment. The density of CD31 positive capillaries in the EES group was significantly higher than those from the BMS and DES groups on day 7, suggesting a pro-angiogenesis role of EES (FIGS. 7G-7J). In addition, EES treatment increased the number of Ki67 positive endothelial cells ((yellow, FIG. 7H). The Ki67 positive cells in the BMS or DES groups on day 28 could be due to the infiltration of monocytes and remodeling. As shown in FIG. 19, the number of CD68 positive cells remained at a high level in both BMS and DES groups. In contrast, infiltration of CD68 positive cells in the EES group dropped to a normal level on day 28.

TABLE 2
Comparison of BMS, DES and EES.

| | BMS | DES | EES |
|---|---|---|---|
| Surface | Metal | Polymer | Phospholipid |
| | | | bilayer |
| Factors | None | Anti-proliferation | Exosomes |
| released | | drugs | |
| Main | Prevent | Reduce in-stent | Biocompatible |
| functions | occlusion | restenosis | Reduce inflammation |
| | | | Repair injured |
| | | | vessels and |
| | | | ischemic tissues |
| Limitations | In-stent | Late and very | Exosomes are |
| | restenosis | late stent | considered |
| | | thrombosis | biologics |
| Possible | Neointimal | Chronic inflammation | |
| causes | hyperplasia | due to the polymer | |
| | during vessel | coating | |
| | healing, and | Suppression of | |
| | inflammation | endothelization | |
| | due to foreign | | |
| | body reaction | | |

What is claimed is:

1. A stent device comprising a plurality of extracellular vesicles conjugated to its surface with a chemical linker.

2. The device of claim 1, wherein the plurality of extracellular vesicles comprise naturally-occurring and/or engineered exosomes, microvesicles, and/or liposomes.

3. The device of claim 2, wherein the plurality of extracellular vesicles are derived from adult stem cells, induced pluripotent stem cells, and/or embryonic stem cells.

4. The device of claim 2 or claim 3, wherein the plurality of extracellular vesicles are derived from mesenchymal stem cells (MSCs), cardiac stem cells (CSCs), cardiac progenitor cells (CPCs), cardiosphere-derived cells (CDCs), hematopoietic stem cells (HSCs), and/or hematopoietic progenitor cells (HPCs).

5. The device of any of claims 1 to 4, wherein the plurality of extracellular vesicles comprise one or more therapeutic microRNAs (miRNAs) selected from the group consisting of hsa-let-7c-5p, hsa-let-7b-5p, hsa-let-7a-5p, hsa-miR-100-5p, hsa-miR-99a-5p, hsa-let-7f-5p, hsa-miR-23b-3p, hsa-miR-23a-3p, hsa-let-7i-5p, hsa-let-7g-5p, hsa-miR-10a-5p, hsa-miR-99b-5p, hsa-miR-148a-3p, hsa-miR-191-5p, hsa-miR-26a-5p, hsa-miR-1290, hsa-miR-320a-3p, hsa-miR-320b, hsa-miR-143-3p, hsa-miR-152-3p, hsa-miR-125b-5p, hsa-let-7d-5p, hsa-miR-320c, hsa-miR-3184-3p, hsa-miR-423-5p, and any combinations thereof.

6. The device of claim 2, wherein the liposomes comprise saturated and unsaturated fatty acid chains suitable for lipid particles.

7. The device of claim 6, wherein the fatty acid chains are selected from the group consisting of: 1,2-Distearoyl-sn-glycero-3-phosphoethanolamine (DSPE); 1,2-Distearoyl-sn-glycero-3-phosphocholine (DSPC); 1-palmitoyl-2-oleoyl-sn-glycero-3-phosphocholine (POPC); N-(2,3-dioleoyloxy) propyl)-N,N,N-triethylammonium chloride (DOTAP); and 3-(N—(N′, N′-dimethylaminoethane)-carbamoyl) cholesterol (DC-Chol).

8. The device of any of claims 1 to 7, wherein the plurality of extracellular vesicles comprise a therapeutic agent.

9. The device of claim 8, wherein the therapeutic agent comprises an anti-platelet drug and/or a regenerative factor.

10. The device of any of claims 1 to 9, wherein the plurality of extracellular vesicles comprise an average size of about 50 nm to about 500 nm.

11. The device of any of claims 1 to 10, wherein the device comprises about 10⁵ to about 10¹⁰ extracellular vesicles per mm².

12. The device of any of claims 1 to 10, wherein the device comprises about 10⁸ to about 10⁹ extracellular vesicles per mm².

13. The device of any of claims 1 to 12, wherein a portion of the linker is sensitive to a cleavage agent, wherein the cleavage agent is capable releasing the plurality of extracellular vesicles from the device.

14. The device of claim 13, wherein the linker comprises a reactive oxygen species (ROS)-sensitive portion, and wherein the cleavage agent is an ROS.

15. The device of any of claims 1 to 14, wherein the linker comprises a phospholipid-polymer conjugate, wherein the plurality of extracellular vesicles are conjugated to the device via the phospholipid-polymer conjugate.

16. The device of claim 15, wherein the phospholipid-polymer conjugate is inserted into the lipid membranes of the plurality of extracellular vesicles.

17. The device of claim 16, wherein the phospholipid-polymer conjugate comprises 1,2-Distearoyl-sn-glycero-3-phosphoethanolamine-Poly(ethylene glycol) (DSPE-PEG) or any related phosphatidylcholine.

18. The device of claim 17, wherein the PEG comprises a molecular weight of about 2,000 to about 20,000.

19. The device of any of claims 1 to 18, wherein the linker comprises thioether, alkyl selenide, telluride, alkyl diselenide, arylboronic ester, carboxyphenylboronic acid, thioketal, polysaccharide, aminoacrylate, oligoproline, and/or peroxalate ester.

20. The device of any of claims 1 to 19, wherein the surface of the device is functionalized.

21. The device of claim 20, wherein the functionalization comprises hydroxylation and/or silanization to generate chemically active groups, such as amino, hydroxyl, thiol and/or carboxy groups.

22. The device of claim 21, wherein the portion of the linker sensitive to the cleavage agent is conjugated to the device via the chemically active groups.

23. A method of treating stenosis, restenosis, and/or ischemic injury comprising implanting the stent of any of claims 1 to 22 into a blood vessel of a subject.

24. The method of claim 23, wherein the ischemic injury comprises myocardial infarction, peripheral artery disease, stroke, mesenteric ischemia and/or renal ischemia.

25. The method of claim 23 or 24, wherein the stent treats the stenosis or restenosis by increasing endothelial cell proliferation and/or inhibiting smooth muscle cell migration.

26. The method of any of claims 23 to 25, wherein the stent treats the ischemia by increasing tissue regeneration.
